SEQUENCE LISTING (11 sequences)

SEQ ID NO: 1 - DNA, 29 bp, Synthetic construct
  source 1..29: mol_type = other DNA
  modified_base 23: mod_base = OTHER; note = uracil
aatgatacggcgaccaccgagatctacac

SEQ ID NO: 2 - DNA, 29 bp, Synthetic construct
  source 1..29: mol_type = other DNA
  modified_base 23: mod_base = OTHER; note = inosine
aatgatacggcgaccaccgaganctacac

SEQ ID NO: 3 - DNA, 29 bp, synthetic construct
  source 1..29: mol_type = other DNA
  modified_base 23: mod_base = OTHER; note = alkene-thymidine
aatgatacggcgaccaccgaganctacac

SEQ ID NO: 4 - DNA, 24 bp, Synthetic construct
  source 1..24: mol_type = other DNA
  modified_base 22: mod_base = OTHER; note = 8-oxoguanine
caagcagaagacggcatacganat

SEQ ID NO: 5 - DNA, 24 bp, Synthetic construct
  source 1..24: mol_type = other DNA
  modified_base 20: mod_base = OTHER; note = 8-oxoguanine
caagcagaagacggcatacnagat

SEQ ID NO: 6 - DNA, 24 bp, Synthetic construct
  source 1..24: mol_type = other DNA
  modified_base 22: mod_base = OTHER; note = 8-oxoguanine
  modified_base 20: mod_base = OTHER; note = 8-oxoguanine
caagcagaagacggcatacnanat

SEQ ID NO: 7 - DNA, 29 bp, Synthetic construct
  source 1..29: mol_type = other DNA
  modified_base 23: mod_base = OTHER; note = a thymine nucleotide analog having an allyl functionality
aatgatacggcgaccaccgaganctacac

SEQ ID NO: 8 - DNA, 35 bp, Synthetic construct
  source 1..35: mol_type = other DNA
gctggcacgtccgaacgcttcgttaatccgttgag

SEQ ID NO: 9 - DNA, 35 bp, Synthetic construct
  source 1..35: mol_type = other DNA
cgtcgtctgccatggcgcttcggtggatatgaact

SEQ ID NO: 10 - DNA, 35 bp, Synthetic construct
  source 1..35: mol_type = other DNA
acggccgctaatatcaacgcgtcgaatccgcaact

SEQ ID NO: 11 - DNA, 34 bp, Synthetic construct
  source 1..34: mol_type = other DNA
gccgcgttacgttagccggactattcgatgcagc

(19) United States
(12) Patent Application Publication
(10) Pub. No.: US 2026/0117218 A1
(43) Pub. Date: Apr. 30, 2026

(54) CONTROLLED DNA FRAGMENT LENGTH BIAS FOR AMPLIFICATION

(71) Applicant: ILLUMINA, INC., San Diego, CA (US)

(72) Inventors: Thomas Gros, Encinitas, CA (US); Michael Jacobs, San Diego, CA (US); Sean Ramirez, San Diego, CA (US)

(21) Appl. No.: 19/366,134

(22) Filed: Oct. 22, 2025

Related U.S. Application Data
(60) Provisional application No. 63/714,546, filed on Oct. 31, 2024.

(51) Int. Cl. C12N 15/10; C12Q 1/6806; C12Q 1/6844

(52) U.S. Cl. CPC C12N 15/1006; C12Q 1/6806; C12Q 1/6844

(57) ABSTRACT

Flow cells and methods are described that enable control over DNA fragment length bias during amplification. One example flow cell includes a substrate and a polymeric hydrogel positioned over at least a portion of the substrate. A plurality of oligonucleotide primers may be attached to the polymeric hydrogel, wherein the plurality of oligonucleotide primers defines a density gradient across the substrate of the flow cell.

### CROSS-REFERENCE TO RELATED APPLICATIONS

This application claims the benefit of U.S. Provisional Application Ser. No. 63/714,546, filed Oct. 31, 2024, the content of which is incorporated by reference herein in its entirety.

### REFERENCE TO SEQUENCE LISTING

The instant application contains a Sequence Listing which has been submitted electronically in XML file format and is hereby incorporated by reference in its entirety. Said XML copy, created on Oct. 20, 2025, is named IL1287B_IP-2826-US_Sequence_Listing.xml and is 15,137 bytes in size.

### BACKGROUND

Double-stranded DNA (dsDNA) target molecules can be fragmented and tagged to generate a library of smaller dsDNA. These smaller dsDNA can be further processed to form single-stranded DNA molecules (ssDNA). These smaller, single-stranded DNA molecules may be used as templates in DNA amplification and sequencing reactions. The templates may enable short read lengths to be obtained, and then during data analysis, overlapping short sequence reads can be aligned to reconstruct the longer nucleic acid sequences. Some methods for fragmentation, tagging, and amplification are limited with regard to control over the size of the resulting fragments.

### SUMMARY

Some biological sequencing operations utilize amplification of strands of DNA, e.g., via the use of a polymerase, to generate clusters of amplicons that are sequenced. It may be desirable to bias the length of the strands (i.e., fragments) that are involved in such amplification processes, thus biasing the insert size of the fragments. The ability to bias the insert size of these strands may be advantageous, e.g., for improving sequencing metrics, reducing pooling bias, and/or for tailoring biological sequencing operations to particular applications, such as fetal blood testing. In some examples, the bias can be shifted in one direction or the other (i.e., to smaller fragments or larger fragments from the average fragment length obtained using a particular method) by about 100 base pairs (bp) on a 700 bp sample, although larger shifts (e.g., from greater than 100 bp to about 300 bp) are also possible.

Disclosed herein are flow cells and methods that can be utilized to bias the length of DNA fragments that are involved in amplification. In some instances, the physical architecture of the flow cell enables the bias. In other instances, the changes to the amplification mix and/or conditions enable the bias. In still other instances, combinations of the flow cell architecture and the amplification mix and/or conditions enable the bias. By biasing longer or shorter fragments to participate in amplification, amplicons with longer or shorter insert sizes are generated, either of which can be beneficial, depending upon the goal.

### BRIEF DESCRIPTION OF THE DRAWINGS

Features of examples of the present disclosure will become apparent by reference to the following detailed description and drawings, in which like reference numerals correspond to similar, though perhaps not identical, components. For the sake of brevity, reference numerals or features having a previously described function may or may not be described in connection with other drawings in which they appear.

FIG. 1A is a top view of an example of a flow cell;

FIG. 1B is an enlarged, and partially cutaway view of an example of a flow cell substrate, where a polymeric hydrogel having primers attached thereto is positioned over the substrate, where the primers form a density gradient across a surface of the substrate, and where a thickness of the polymeric hydrogel is varied to define distinct regions of the density gradient;

FIG. 1C is an enlarged, and partially cutaway view of an example of a flow cell substrate including a plurality of depressions defined therein that are separated by interstitial regions, where a polymeric hydrogel having primers attached thereto is positioned in each of the depressions, where the primers form a density gradient across a surface of the substrate, and where a diameter of the depressions is varied to define distinct regions of the density gradient;

FIG. 1D is an enlarged, and partially cutaway view of an example of a flow cell substrate, where a polymeric hydrogel having primers attached thereto is positioned over the substrate, where the primers form a density gradient across a surface of the substrate, and where a density of polymeric hydrogel functional groups is varied across a surface of the substrate to define distinct regions of the density gradient;

FIG. 1E is an enlarged, and partially cutaway view of an example of a flow cell substrate including depressions separated by interstitial regions, and a polymeric hydrogel having primers attached thereto in each of the depressions;

FIG. 2A through FIG. 2D collectively and schematically show an example of a method of preparing a flow cell substrate, where FIG. 2A depicts an unpatterned flow cell substrate, FIG. 2B depicts the structure of FIG. 2A after a polymeric hydrogel has been applied thereon, where a thickness of the polymeric hydrogel is varied across the substrate, FIG. 2C depicts the structure of FIG. 2B after a polishing process has been performed, and FIG. 2D depicts the structure of FIG. 2C after primers have been grafted thereto and a density gradient has been formed;

FIG. 3A through FIG. 3D are schematic illustrations that collectively show an example of a method of preparing a flow cell substrate, where FIG. 3A depicts a flow cell having a plurality of depressions defined therein, where individual depressions in the plurality of depressions have at least two different diameters at distinct regions of the substrate, FIG. 3B depicts the structure of FIG. 3A after a polymeric hydrogel has been applied thereon, FIG. 3C depicts the structure of FIG. 3B after a polishing process has been performed, and FIG. 3D depicts the structure of FIG. 3C after primers have been grafted to the polymeric hydrogel within the depressions and a density gradient has been formed;

FIG. 4A through FIG. 4D are schematic illustrations that collectively show an example of a method of preparing a flow cell substrate, where FIG. 4A depicts a flow cell substrate having bonding regions, FIG. 4B depicts the structure of FIG. 4A after a polymeric hydrogel has been applied thereon, wherein distinct regions of the polymeric hydrogel have different functional group densities with respect to one another, FIG. 4C depicts the structure of FIG. 4B after a polishing process has been performed, and FIG. 4D depicts the structure of FIG. 4C after primers have been grafted to the polymeric hydrogel and a density gradient has been formed;

FIG. 5A through FIG. 5E are schematic illustrations that collectively show an example of a method, where FIG. 5A depicts library fragments seeded in a depression of a flow cell, FIG. 5B depicts the compaction of the seeded library fragments of FIG. 5A after a crowding agent solution is introduced, FIG. 5C depicts the release of previously seeded smaller library fragments during simultaneous heating and introduction of additional crowding agent solution, FIG. 5D depicts remaining seeded (larger) library fragments as the temperature is lowered, and FIG. 5E depicts the remaining seeded library fragments as a buffer solution is introduced;

FIG. 6 is a graph depicting the results of an experiment that was performed to ascertain the effect of primer density on resultant fragment length after amplification, where primer density is shown on the Y axis and fragment length (in terms of the number of base pairs) is shown on the X axis; and

FIG. 7 is a graph depicting the results of an experiment that was performed to ascertain the effect of primer availability on resultant fragment length after amplification, where % passing filter is shown on the Y axis and library type (in terms of average fragment length) is shown on the X axis.

### DETAILED DESCRIPTION

The examples disclosed herein enable a user to bias the length of DNA fragments that are involved in amplification, and in turn, sequencing. By biasing longer fragments to participate in amplification, longer amplicons are generated. Longer amplicons help when aligning reads back to a reference, as the forward strand reads and reverse strand reads (e.g., read 1 and read 2) will be further apart. Longer amplicons can also reduce the chance of read 1 and read 2 overlapping, which can help with GC bias and improve sequencing accuracy of otherwise tricky regions (e.g., short tandem repeats (STRs), homopolymers, etc.). Biasing for short fragments may be desirable when sequencing data for a particular type of genetic material within a sample is of interest. For example, biasing for short fragments may be desirable when the sample includes both fetal DNA and maternal DNA, or when a sewage sample includes both bacterial and viral genomes.

### Definitions

It is to be understood that terms used herein will take on their ordinary meaning in the relevant art unless specified otherwise. Several terms used herein and their meanings are set forth below.

The singular forms “a”, “an”, and “the” include plural referents unless the context clearly dictates otherwise.

The terms comprising, including, containing and various forms of these terms are synonymous with each other and are meant to be equally broad.

The terms top, bottom, lower, upper, on, adjacent, etc. are used herein to describe the flow cell and/or the various components of the flow cell. It is to be understood that these directional terms are not meant to imply a specific orientation, but are used to designate relative orientation between components. The use of directional terms should not be interpreted to limit the examples disclosed herein to any specific orientation(s).

The terms first, second, etc. also are not meant to imply a specific orientation or order, but rather are used to distinguish one component from another.

It is to be understood that the ranges provided herein include the stated range and any value or sub-range within the stated range, as if such values or sub-ranges were explicitly recited. For example, a range of about 630 nm to about 700 nm should be interpreted to include not only the explicitly recited limits of about 630 nm to about 700 nm, but also to include individual values, such as about 650 nm, 675.5 nm, 699 nm, etc., and sub-ranges, such as from about 650 nm to about 690 nm, from about 655 nm to about 680 nm, etc.

An “acrylamide monomer” refers to a monomer with the structure

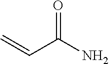

or a monomer including an acrylamide group. Examples of the monomer including an acrylamide group include azido acetamido pentyl acrylamide:

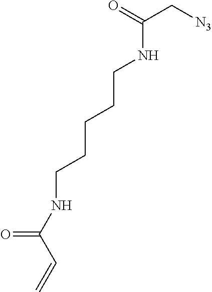 [SMILES: C=CC(=O)NCCCCCNC(=O)CN=[N+]=[N-]]

### and N-isopropylacrylamide:

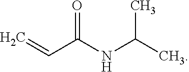

Other acrylamide monomers may be used.

The term “activation,” as used herein, refers to a process that generates reactive groups at the surface of a single-layer substrate or an outermost layer of a multi-layer substrate. Activation may be accomplished using silanization (e.g., exposure to a silane-inclusive solution or mixture) and/or plasma ashing.

An “aldehyde,” as used herein, refers to an organic compound containing a functional group with the structure —CHO, which includes a carbonyl center (i.e., a carbon double-bonded to oxygen), with the carbon atom also being bonded to hydrogen and an R group (such as an alkyl or other side chain). The general structure of an aldehyde is:

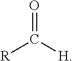

As used herein, “alkyl” refers to a straight or branched hydrocarbon chain that is fully saturated (i.e., contains no double or triple bonds). The alkyl group may have 1 to 20 carbon atoms. Example alkyl groups include methyl, ethyl, propyl, isopropyl, butyl, isobutyl, tertiary butyl, pentyl, hexyl, and the like. As an example, the designation “C1-4 alkyl” indicates that there are one to four carbon atoms in the alkyl chain, i.e., the alkyl chain is selected from the group consisting of methyl, ethyl, propyl, iso-propyl, n-butyl, isobutyl, sec-butyl, and tert-butyl.

As used herein, “alkenyl” refers to a straight or branched hydrocarbon chain containing one or more double bonds. The alkenyl group may have 2 to 20 carbon atoms. Example alkenyl groups include ethenyl, propenyl, butenyl, pentenyl, hexenyl, and the like.

As used herein, “alkyne” or “alkynyl” refers to a straight or branched hydrocarbon chain containing one or more triple bonds. The alkynyl group may have 2 to 20 carbon atoms.

As used herein, “aryl” refers to an aromatic ring or ring system (i.e., two or more fused rings that share two adjacent carbon atoms) containing only carbon in the ring backbone. When the aryl is a ring system, every ring in the system is aromatic. The aryl group may have 6 to 18 carbon atoms. Examples of aryl groups include phenyl, naphthyl, azulenyl, and anthracenyl.

An “amino” functional group refers to an —NRₐR_{b} group, where Rₐ and R_{b} are each independently selected from hydrogen

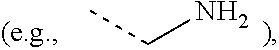

C1-6 alkyl, C2-6 alkenyl, C2-6 alkynyl, C3-7 carbocycle, C6-10 aryl, 5-10 membered heteroaryl, and 5-10 membered heterocycle, as defined herein.

As used herein, the term “attached” refers to the state of two things being joined, fastened, adhered, connected, or bound to each other, either directly or indirectly, and either physically or chemically. As an example of chemical attachment, a nucleic acid can be attached to a polymeric hydrogel by a covalent or non-covalent bond. A covalent bond is characterized by the sharing of pairs of electrons between atoms. A non-covalent bond is a physical bond that does not involve the sharing of pairs of electrons and can include, for example, hydrogen bonds, ionic bonds, van der Waals forces, hydrophilic interactions, and hydrophobic interactions.

An “azide” or “azido” functional group refers to —N₃.

As used herein, a “bonding region” refers to an area of a substrate that is to be bonded to another material, which may be, as examples, a spacer layer, a lid, another substrate, etc., or combinations thereof (e.g., a spacer layer and a lid, or a spacer layer and another substrate). The bond that is formed at the bonding region may be a chemical bond (as described above), or a mechanical bond (e.g., using a fastener, etc.). The bonding region is free of surface chemistry (e.g., polymeric hydrogel and primers of a primer set).

As used herein, “carbocycle” means a non-aromatic cyclic ring or ring system containing only carbon atoms in the ring system backbone. When the carbocycle is a ring system, two or more rings may be joined together in a fused, bridged, or spiro-connected fashion. Carbocycles may have any degree of saturation, provided that at least one ring in a ring system is not aromatic. Thus, carbocycles include cycloalkyls, cycloalkenyls, and cycloalkynyls. The carbocycle group may have 3 to 20 carbon atoms. Examples of carbocycle rings include cyclopropyl, cyclobutyl, cyclopentyl, cyclohexyl, cyclohexenyl, 2,3-dihydro-indene, bicyclo[2.2.2]octanyl, adamantyl, and spiro[4.4]nonanyl.

As used herein, the term “carboxylic acid” or “carboxyl” refers to —COOH.

As used herein, “cycloalkyl” refers to a completely saturated (no double or triple bonds) mono- or multi-cyclic hydrocarbon ring system. When composed of two or more rings, the rings may be joined together in a fused fashion. Cycloalkyl groups can contain 3 to 10 atoms in the ring(s). In some examples, cycloalkyl groups can contain 3 to 8 atoms in the ring(s). A cycloalkyl group may be unsubstituted or substituted. Example cycloalkyl groups include cyclopropyl, cyclobutyl, cyclopentyl, cyclohexyl, cycloheptyl, and cyclooctyl.

As used herein, “cycloalkenyl” or “cycloalkene” means a carbocycle ring or ring system having at least one double bond, wherein no ring in the ring system is aromatic. Examples include cyclohexenyl or cyclohexene and norbornenyl or norbornene. Also as used herein, “heterocycloalkenyl” or “heterocycloalkene” means a carbocycle ring or ring system with at least one heteroatom in ring backbone, having at least one double bond, wherein no ring in the ring system is aromatic.

As used herein, “cycloalkynyl” or “cycloalkyne” means a carbocycle ring or ring system having at least one triple bond, wherein no ring in the ring system is aromatic. An example is cyclooctyne. Another example is bicyclononyne. Also as used herein, “heterocycloalkynyl” or “heterocycloalkyne” means a carbocycle ring or ring system with at least one heteroatom in ring backbone, having at least one triple bond, wherein no ring in the ring system is aromatic.

The term “density gradient,” as used herein, refers to a substrate that includes at least two different densities of primers in two different areas (i.e., at least two different primer densities across the substrate). “Primer density,” as used herein, refers to an approximate number of oligonucleotide primers that are attached, e.g., to a polymeric hydrogel, within a given area of the substrate, and the term may be defined in terms of primers/nm². The density gradient may have a pattern, which encompasses any number of distinct regions of differing (and in some instances, progressively increasing) primer densities. For example, the pattern may include two distinct regions, three distinct regions, four distinct regions, five distinct regions, etc., of the polymeric hydrogel, where each region has a unique primer density with respect to the other region(s).

The term “depositing,” as used herein, refers to any suitable application technique, which may be manual or automated, and, in some instances, results in modification of the surface properties. Generally, depositing may be performed using vapor deposition techniques, coating techniques, grafting techniques, or the like. Some specific examples include chemical vapor deposition (CVD), spray coating (e.g., ultrasonic spray coating), spin coating, dunk or dip coating, doctor blade coating, puddle dispensing, flow through coating/deposition, aerosol printing, screen printing, microcontact printing, inkjet printing, or the like.

As used herein, the term “depression” refers to a discrete concave or recessed feature defined in a substrate and having a surface opening. In some instances, the surface opening is at least partially surrounded by interstitial region(s) of the substrate. Depressions can have any of a variety of shapes at their opening in a surface including, as examples, round, elliptical, square, polygonal, star shaped (with any number of vertices), etc. The cross-section of a depression taken orthogonally with the surface can be curved, square, polygonal, hyperbolic, conical, angular, etc. As examples, the depression can be a well or two interconnected wells.

The term “epoxy” as used herein refers to

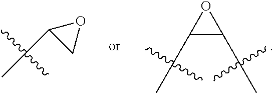

As used herein, the term “flow cell” is intended to refer to a vessel having an enclosed flow channel where a reaction can be carried out, or a vessel that is open to a surrounding environment and in which a reaction can be carried out. A flow cell with an enclosed channel also includes an inlet for delivering reagent(s) to the channel and an outlet for removing reagent(s) from the channel. In some examples, the flow cell enables the detection of the reaction that occurs therein. For example, the flow cell can include one or more transparent surfaces allowing for the optical detection of arrays, optically labeled molecules, or the like.

As used herein, a “flow channel,” “channel,” or “lane” refers to a flow cell area that can selectively receive a liquid sample, reagents, etc. In some examples disclosed herein, the terms refer to an area that is defined between two patterned structures, and the flow channel (or lane) is in fluid communication with surface chemistry disposed within depressions on either of the two substrates. In other examples disclosed herein, the flow channel is defined between one substrate and a lid, and the flow channel is in fluid communication with surface chemistry within the one substrate.

“Fragments” refer to pieces of a larger DNA strand (e.g., such as those found in a gross sample. “Smaller fragments” refer to those that are 200 bp or less, and “longer fragments” refer to those that are greater than 200 bp. In one specific example, the bias may be shifted to target shorter fragments of 150 bp. In another example, the bias may be shifted to target longer fragments of 350 bp.

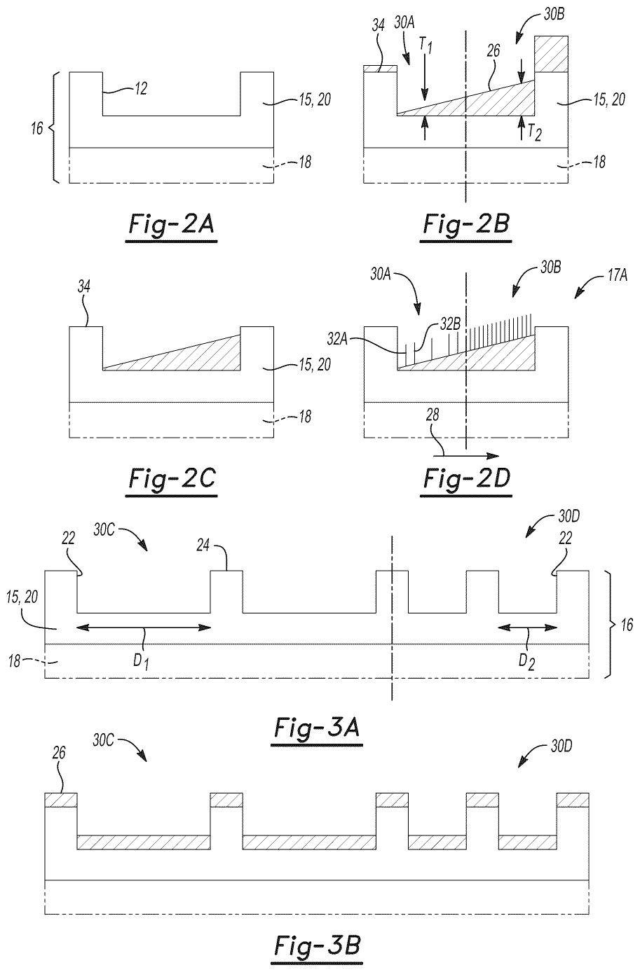

The term “functional group density,” as used herein, refers to an approximate number of a particular type of functional group within a predetermined surface area of a material (e.g., a polymeric hydrogel, a substrate, etc.). For example, the term may refer to an approximate number of azides that are present within a region of a polymeric hydrogel. In some examples described herein, such as that shown in FIG. 2D, a polymeric hydrogel having different functional group densities at different regions may be used to form a density gradient that is used to control fragment bias during amplification.

As used herein, “heteroaryl” refers to an aromatic ring or ring system (i.e., two or more fused rings that share two adjacent atoms) that contain(s) one or more heteroatoms, that is, an element other than carbon, including but not limited to, nitrogen, oxygen and sulfur, in the ring backbone. When the heteroaryl is a ring system, every ring in the system is aromatic. The heteroaryl group may have 5-18 ring members.

As used herein, “heterocycle” means a non-aromatic cyclic ring or ring system containing at least one heteroatom in the ring backbone. Heterocycles may be joined together in a fused, bridged, or spiro-connected fashion. Heterocycles may have any degree of saturation provided that at least one ring in the ring system is not aromatic. In the ring system, the heteroatom(s) may be present in either a non-aromatic or aromatic ring. The heterocycle group may have 3 to 20 ring members (i.e., the number of atoms making up the ring backbone, including carbon atoms and heteroatoms). In some examples, the heteroatom(s) are O, N, or S.

The term “hydrazine” or “hydrazinyl” as used herein refers to a —NHNH₂ group.

The term “hydrazone” or “hydrazonyl,” as used herein, refers to a

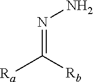

group in which Rₐ and R_{b} are each independently selected from hydrogen, C1-6 alkyl, C2-6 alkenyl, C2-6 alkynyl, C3-7 carbocycle, C6-10 aryl, 5-10 membered heteroaryl, and 5-10 membered heterocycle, as defined herein.

As used herein, “hydroxy” or “hydroxyl” refers to an —OH group.

The term “hydrogel” or “polymeric hydrogel” refers to a semi-rigid polymer that is permeable to liquids and/or gases. The hydrogel can swell when liquid (e.g., water) is taken up and that can contract when liquid is removed, e.g., by drying. While a hydrogel may absorb water, it is not water-soluble.

The “insert size” refers to the length of a library template (i.e., DNA library fragment), without its appended adapters, that is to be used in a sequencing operation.

As used herein, the term “interstitial region” refers to an area, e.g., of a substrate that separates individual depressions from immediately adjacent depressions or from bonding regions. The separation provided by an interstitial region can be partial or full separation.

“Nitrile oxide,” as used herein, means a “RₐC≡N⁺O⁻” group in which Rₐ is defined herein. Examples of preparing nitrile oxide include in situ generation from aldoximes by treatment with chloramide-T or through action of base on imidoyl chlorides [RC(Cl)═NOH] or from the reaction between hydroxylamine and an aldehyde.

“Nitrone,” as used herein, means a

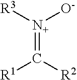

group in which R¹, R², and R³ may be any of the Rₐ and R_{b} groups defined herein, except that R³ is not hydrogen (H).

A “non-patterned structure” refers to a substrate that includes primers that are not present in a pattern.

As used herein, a “nucleotide” includes a nitrogen-containing heterocyclic base, a sugar, and one or more phosphate groups. Nucleotides are monomeric units of a nucleic acid sequence. In RNA (ribonucleic acid), the sugar is a ribose, and in DNA (deoxyribonucleic acid), the sugar is a deoxyribose, i.e., a sugar lacking a hydroxyl group that is present at the 2′ position in ribose. The nitrogen containing heterocyclic base (i.e., nucleobase) can be a purine base or a pyrimidine base. Purine bases include adenine (A) and guanine (G), and modified derivatives or analogs thereof. Pyrimidine bases include cytosine (C), thymine (T), and uracil (U), and modified derivatives or analogs thereof. The C-1 atom of deoxyribose is bonded to N-1 of a pyrimidine or N-9 of a purine. A nucleic acid analog may have any of the phosphate backbone, the sugar, or the nucleobase altered. Examples of nucleic acid analogs include, for example, universal bases or phosphate-sugar backbone analogs, such as peptide nucleic acid (PNA).

In some examples, the term “over” may mean that one component or material is positioned directly on another component or material. When one is directly on another, the two are in physical contact with each other.

In other examples, the term “over” may mean that one component or material is positioned indirectly on another component or material. By indirectly on, it is meant that a gap or an additional component or material may be positioned between the two components or materials.

A “patterned structure” refers to a substrate that includes primers in a pattern. In some examples, the patterned structure includes a pattern of depressions separated by interstitial regions, and primers within the depressions. In other examples, the patterned structure includes a primer density gradient across the surface.

As used herein, the term “polyhedral oligomeric silsesquioxane” (an example of which is commercially available under the tradename “POSS”) refers to a chemical composition that is a hybrid intermediate (e.g., RSiO_{1.5}) between that of silica (SiO₂) and silicone (R₂SiO). An example of polyhedral oligomeric silsesquioxane may be that described in Kehagias et al., Microelectronic Engineering 86 (2009), pp. 776-778, which is incorporated by reference in its entirety. In an example, the composition is an organosilicon compound with the chemical formula [RSiO_{3/2}]ₙ, where the R groups can be the same or different. Example R groups for POSS include epoxy, azide/azido, a thiol, a poly(ethylene glycol), a norbornene, a tetrazine, acrylates, and/or methacrylates, or further, for example, alkyl, aryl, alkoxy, and/or haloalkyl groups.

As used herein, the “primer” is defined as a single stranded nucleic acid sequence (e.g., single stranded DNA). Some primers, referred to herein as amplification primers, serve as a starting point for template amplification and cluster generation. Other primers, referred to herein as sequencing primers, serve as a starting point for DNA synthesis. The 5′ terminus of the primer may be modified to allow a coupling reaction with a functional group of a polymeric hydrogel. The primer length can be any number of bases long and can include a variety of non-natural nucleotides. In an example, the sequencing primer is a short strand, ranging from 10 to 60 bases, or from 20 to 40 bases.

A “spacer layer,” as used herein refers to a material that bonds two components together. In some examples, the spacer layer can be a radiation-absorbing material that aids in bonding, or can be put into contact with a radiation-absorbing material that aids in bonding.

The term “substrate” may be used herein in conjunction with the term “single-layer substrate” or “multi-layer substrate.” A single-layer substrate is one layer of a support material that can be used to form a patterned structure (referred to herein as a “single-layer substrate 15” or “substrate 15”). The multi-layer substrate includes at least two layers, e.g., a base support with an additional layer thereon that can be patterned with depressions (sometimes referred to herein as a “multi-layer substrate 16” or “substrate 16,” which includes the base support 18 and the layer 20 thereon).

“Surface chemistry,” as defined herein, refers to a hydrophilic material, such as a polymeric hydrogel (as defined herein), which may have at least one primer attached thereto.

The term “tantalum pentoxide” refers to the inorganic compound with the formula Ta₂O₅. This compound is transparent, having a transmittance ranging from about 0.25 (25%) to 1 (100%), to wavelengths ranging from about 0.35 μm (350 nm) to at least 1.8 μm (1800 nm). A “tantalum pentoxide substrate” may comprise, consist essentially of, or consist of Ta₂O₅. In examples where it is desirable for the tantalum pentoxide substrate to transmit electromagnetic energy having any of these wavelengths, the substrate may consist of Ta₂O₅ or may comprise or consist essentially of Ta₂O₅ and other components that will not interfere with the desired transmittance of the substrate.

A “thiol” functional group refers to —SH.

As used herein, the terms “tetrazine” and “tetrazinyl” refer to six-membered heteroaryl group comprising four nitrogen atoms. Tetrazine can be optionally substituted.

“Tetrazole,” as used herein, refers to five-membered heterocyclic group including four nitrogen atoms. Tetrazole can be optionally substituted.

The term “transparent” when describing a material (e.g., substrate, layer, etc.) means that that the material allows light of a particular wavelength or range of wavelengths to pass through. Transparency may be quantified using transmittance, i.e., the ratio of light energy falling on a body to that transmitted through the body. The transmittance of a transparent material will depend upon the thickness of the material and the wavelength of light. In the examples disclosed herein, the transmittance of the transparent material may range from 0.25 (25%) to 1 (100%). The material may be a pure material, a material with some impurities, or a mixture of materials, as long as the resulting material is capable of the desired transmittance. Additionally, depending upon the transmittance of the material, the time for light exposure and/or the output power of the light source may be increased or decreased to deliver a suitable dose of light energy through the transparent material to achieve the desired effect.

### Flow Cells

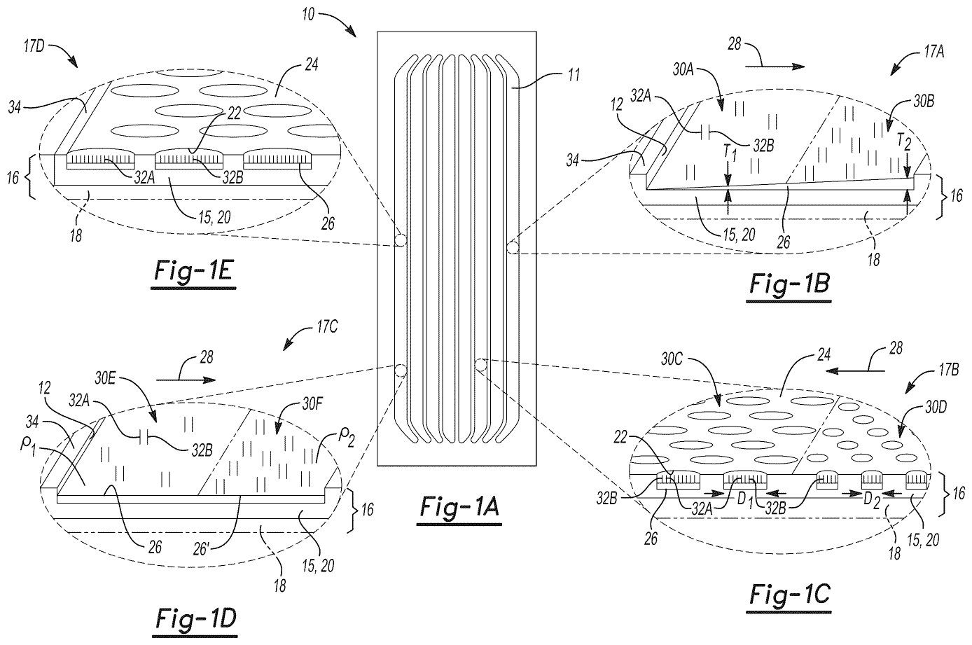

Flow cells are used in conjunction with the methods disclosed herein. In some instances, the flow cell structure itself is used to bias the length of the strands that are to be amplified (and ultimately sequenced) in the flow cell. Each of these flow cell structures includes a primer density gradient. Different examples of the architecture used to achieve the density gradient are shown in FIG. 1B through FIG. 1D. In other instances, the method is used to bias the length of the strands that are to be amplified (and ultimately sequenced) in the flow cell, and the flow cell structure does not include the primer density gradient. Examples of these flow cells are described in reference to FIG. 1D and are shown in FIG. 1E.

Some examples of the flow cell disclosed herein include a substrate, a polymeric hydrogel positioned over at least a portion of the substrate, and a plurality of oligonucleotide primers attached to the polymeric hydrogel, wherein the plurality of oligonucleotide primers defines a density gradient across the substrate.

FIG. 1A depicts an example of the flow cell 10 disclosed herein from a top view, and different architectures that may be included within individual flow channels 11 of the flow cell 10 are respectively shown in FIG. 1B through FIG. 1E. FIG. 1B through FIG. 1E depict different patterned structures 17A-17D, each of which includes a substrate 15 or 16.

The pattered structure 17A of FIG. 1B includes the substrate 15, 16, which has a lane 12 defined therein. In this example, a polymeric hydrogel 26 is positioned within the lane 12, and the polymeric hydrogel 26 has a thickness that varies at distinct regions of the substrate 15, 16 (see, e.g., the different thicknesses T₁, T₂ at the different regions 30A, 30B of the substrate 15, 16 in FIG. 1B). The different thicknesses T₁, T₂ of the polymeric hydrogel 26 layer in FIG. 1B define distinct regions 30A, 30B of a density gradient 28, as will be described in more detail herein.

The patterned structure 17B of FIG. 1C includes the substrate 15, 16, which has a plurality of depressions 22 separated by interstitial regions 24. As shown in the figure, at least two distinct regions 30C, 30D of the substrate 15, 16 respectively include depressions 22 having different diameters D₁, D₂. The distinct regions 30C, 30D help define the density gradient 28, as will be described in more detail herein.

The patterned structure 17C of FIG. 1D includes the substrate 15, 16, which has the lane 12 defined therein. In this example, polymeric hydrogels 26, 26′ are positioned at distinct regions 30E, 30F within the lane 12, and the polymeric hydrogels 26, 26′ have different functional group densities ρ₁, ρ₂. The different functional group densities ρ₁, ρ₂ help define the density gradient 28, as will be described in more detail herein.

The substrate 15, 16 that has the lane 12 defined therein may also be used to form a non-patterned structure. In the non-patterned structure, a single polymeric hydrogel 26 is present in the lane, and the primers 32A, 32B are grafted across the polymeric hydrogel 26 randomly. The primers 32A, 32B do not form a gradient. This flow cell 10 is suitable for use in the methods described herein that use the amplification mix and/or amplification conditions to create the bias.

The patterned structure 17D of FIG. 1E is also suitable for use in the methods described herein that use the amplification mix and/or amplification conditions to create the bias. The patterned structure 17D includes the substrate 15, 16, which has a plurality of depressions 22 separated by interstitial regions 24. In this example, the primers 32A, 32B are distributed randomly, but relatively evenly, across the depressions 22, and thus there is not a primer density gradient.

Enclosed examples of the flow cell 10 disclosed herein may include one patterned structure 17A, 17B, 17C, or 17D or non-patterned structure bonded to a lid, e.g., at one or more bonding regions 34 (lid not shown in FIG. 1A through FIG. 1E), or one patterned structure 17A, 17B, 17C, or 17D bonded to a second patterned structure (second patterned structure not shown), or one non-patterned structure bonded to a second non-patterned structure. In the enclosed versions of the flow cell 10, the patterned structure 17A, 17B, 17C, or 17D or non-patterned structure may be bonded to the lid or to the second patterned structure or second non-patterned structure via a spacer layer positioned at the bonding region(s) 34. Open-wafer examples of the flow cell 10 include a single patterned structure 17A, 17B, 17C, or 17D or a single non-patterned structure that is open to a surrounding environment.

In enclosed versions of the flow cell 10, the spacer layer that is used to attach the structures or structure and lid may be any material that will seal portions of the structures or structure and lid. As examples, the spacer layer may be an adhesive, a radiation-absorbing material that aids in bonding, or the like. In some examples, the spacer layer is the radiation-absorbing material, e.g., KAPTON® black. The spacer layer may have any suitable thickness, depending upon the desired dimensions of the flow channel 11.

Each patterned structure 17A, 17B, 17C, 17D and non-patterned structure includes the substrate 15 or 16, which may be a single-layer substrate 15 or a multi-layer substrate 16. The multi-layer substrate 16 includes a base support 18 and an additional layer 20 positioned directly over the base support 18.

Examples of suitable materials for the single-layer substrate 15 or the base support 18 include siloxanes (e.g., epoxy siloxane), glass, modified or functionalized glass, polymeric materials (including acrylics, polystyrene and copolymers of styrene and other materials, polypropylene, polyethylene, polybutylene, polyurethanes, polytetrafluoroethylene (such as TEFLON® from Chemours), polyethylene terephthalate (PET), polycarbonate, cyclic olefins/cyclo-olefin polymers (COP) (such as ZEONOR® from Zeon), polyimides, nylon (polyamides), etc.), ceramics/ceramic oxides, silica (i.e., silicon dioxide (SiO₂)), fused silica, or silica-based materials, aluminum silicate, silicon and modified silicon (e.g., boron doped p+ silicon), silicon nitride (Si₃N₄), tantalum pentoxide (Ta₂O₅) or other tantalum oxide(s) (TaOₓ), hafnium oxide (HfO₂), carbon, metals, resins, or the like. Examples of suitable resins include inorganic oxides, such as tantalum pentoxide (e.g., Ta₂O₅) or other tantalum oxide(s) (TaOₓ), aluminum oxide (e.g., Al₂O₃), silicon oxide (e.g., SiO₂), hafnium oxide (e.g., HfO₂), indium tin oxide, titanium dioxide, etc., or polymeric resins, such as a polyhedral oligomeric silsesquioxane based resin (e.g., POSS® from Hybrid Plastics), a non-polyhedral oligomeric silsesquioxane epoxy resin, a poly(ethylene glycol) resin, a polyether resin (e.g., ring-opened epoxies), an acrylic resin, an acrylate resin, a methacrylate resin, an amorphous fluoropolymer resin (e.g., CYTOP® from Bellex), and combinations thereof.

It is to be understood that the material of the single-layer substrate 15 may be any material that can be coated with a polymeric hydrogel 26, 26′ and/or that can be imprinted to form the lane 12 or the depressions 22.

As mentioned, examples of the multi-layer substrate 16 include the base support 18 and at least one other layer 20 positioned thereon. Examples of suitable materials for the layer 20 include inorganic oxides, such as tantalum oxide (e.g., Ta₂O₅), aluminum oxide (e.g., Al₂O₃), silicon oxide (e.g., SiO₂), or hafnium oxide (e.g., HfO₂), or polymeric resins, such as a polyhedral oligomeric silsesquioxane based resin (e.g., POSS® from Hybrid Plastics), a non-polyhedral oligomeric silsesquioxane epoxy resin, a poly(ethylene glycol) resin, a polyether resin (e.g., ring-opened epoxies), an acrylic resin, an acrylate resin, a methacrylate resin, an amorphous fluoropolymer resin (e.g., CYTOP® from Bellex), and combinations thereof. It is to be understood that in examples of the flow cell 10 that include the multi-layer substrate 16, the other layer 20 (positioned on the base support 18) may be any material that can be patterned with a polymeric hydrogel 26 and/or that can be imprinted to form the lane 12 or the depressions 22.

Suitable deposition techniques for the material of the single-layer substrate 15 or for the material(s) of the components of the multi-layer substrate 16 (e.g., the base support 18 and/or the layer 20) include chemical vapor deposition, dip coating, dunk coating, spin coating, spray coating, puddle dispensing, ultrasonic spray coating, doctor blade coating, aerosol printing, screen printing, microcontact printing, etc. It is to be understood that the deposition technique(s) that is/are used may depend, in part, upon the material of the substrate 18 or the material of the components of the substrate 20.

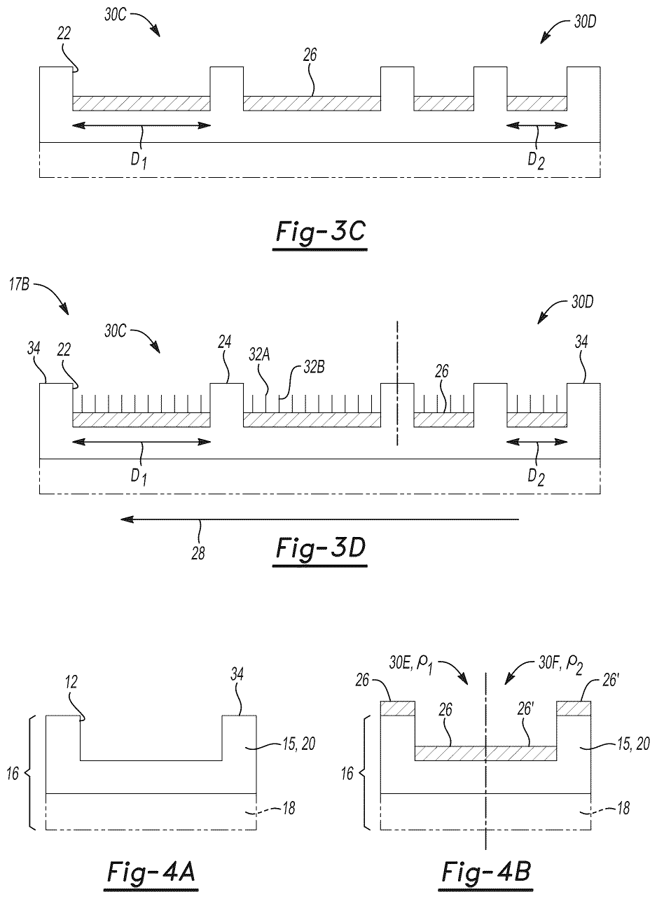

Suitable patterning techniques for the material of the single-layer substrate 15 or for the layer 20 of the multi-layer substrate 16 include photolithography, nanoimprint lithography (NIL), stamping techniques, embossing techniques, molding techniques, micro-etching techniques, etc. It is to be understood that the patterning technique(s) that is/are used may depend, in part, upon the material used for the single-layer substrate 15 or for the layer 20 of the multi-layer substrate 16 and the desired patterned structure 17A, 17B, 17C, 17D that is to be generated. A specific example of a method of forming the depressions 22 is described in more detail in regard to the method of FIG. 3A through FIG. 3D.

The single-layer substrate 15 or the base support 18 may be a circular sheet, a panel, a wafer, a die etc. having a diameter ranging from about 2 mm to about 300 mm, e.g., from about 200 mm to about 300 mm, or may be a rectangular sheet, panel, wafer, die etc. having its largest dimension up to about 10 feet (˜3 meters). For example, a die may have a width ranging from about 0.1 mm to about 10 mm. While example dimensions have been provided, it is to be understood that a single-layer substrate 15 or base support 18 with any suitable dimensions may be used.

The substrate 15, 16 may have any suitable thickness. In examples that include the lane 12, the thickness of the single-layer substrate 15 or of the layer 20 is greater than a depth of the lane 12. In examples that include depressions 22, the thickness of the single-layer substrate 15 or of the layer 20 is greater than a depth of the depressions 22.

As mentioned, the enclosed flow cells 10 include a flow channel 11. In the enclosed flow cells 10, the flow channel(s) 11 is/are defined between the one patterned structure 17A, 17B, 17C, 17D or one non-patterned structure and the lid, or between the one patterned structure 17A, 17B, 17C, 17D and the second patterned structure, or between two non-patterned structures, any of which are bonded together via the spacer layer at one or more bonding region(s) 34. Thus, the flow channel(s) 11 in the enclosed form of the flow cell 10 is/are defined by the patterned structure 17A, 17B, 17C, 17D or non-patterned structure, the spacer layer, and either the lid or the second patterned or non-patterned structure. Alternatively, examples of the open-wafer flow cell 10 include the single patterned structure 17A, 17B, 17C, 17D or the single non-patterned structure. In these examples, the single-layer substrate 15 or the multi-layer substrate 16 that forms the patterned structure 17A, 17B, 17C, 17D or non-patterned structure is open to a surrounding environment, and the flow channel 11 is defined by portions of the patterned structure 17A, 17B, 17C, 17D or portions of the non-patterned structure that includes surface chemistry.

The flow channel(s) 11 may have any desirable shape. In an example, the flow channel 11(s) has/have a substantially rectangular configuration with curved ends, as shown in FIG. 1A. The length and width of the flow channel(s) 11 depends, in part, upon the size of the substrate 15, 16 that is used to form the patterned structure 17A, 17B, 17C, 17D or the non-patterned structure.

The depth of the flow channel(s) 11 in the enclosed versions of the flow cell 10 can be as small as a monolayer thick when microcontact, aerosol, or inkjet printing is used to deposit a separate material (e.g., the spacer layer) that defines at least a portion of the sidewalls of the flow channel 11. This depth could be thicker if the spacer layer is pre-formed or applied via another technique. For other examples, the depth of the flow channel(s) 11 can be about 1 μm, about 10 μm, about 50 μm, about 100 μm, or more. In an example, the depth may range from about 10 μm to about 400 μm. In another example, the depth may range from about 10 μm to about 30 μm. In still another example, the depth is about 5 μm or less. It is to be understood that the depth of the flow channel(s) 11 may be greater than, less than or between the values specified above.

Each flow channel 11 that is included in enclosed versions of the flow cell 10 may be in fluid communication with one or more inlet(s) and outlet(s) (inlet(s) and outlet(s) not shown in FIG. 1A through FIG. 1E). The inlet(s) and outlet(s) of the respective flow channels 11 may be positioned anywhere along the length and width of the flow channel 11 that enables desirable fluid flow, such as at opposed ends of the flow channel 11.

In the enclosed versions of the flow cell 10, the inlet(s) allows fluid(s) to be introduced into the flow channel(s) 11, and the outlet(s) allow(s) fluid(s) to be extracted from the flow channel(s) 11. Each of the inlet(s) and outlet(s) is/are fluidly connected to a fluidic control system (including, e.g., reservoirs, pumps, valves, waste containers, and the like) that controls fluid introduction and expulsion. Some examples of the fluids that may be introduced into the flow channel(s) 11 include reaction components (e.g., DNA sample, polymerases, sequencing primers, nucleotides, etc.), washing solutions, deblocking agents, etc.

Each example of the flow cell 10 disclosed herein includes the polymeric hydrogel 26 or 26 and 26′, which has primers 32A, 32B (e.g., of a primer set) attached thereto. In the patterned structure 17A of FIG. 1B, the polymeric hydrogel 26 forms a layer that is positioned across the lane 12, and the polymeric hydrogel 26 layer has different thicknesses T₁, T₂ in separate, distinct regions 30A, 30B of the substrate 15, 16. In the patterned structure 17B of FIG. 1C, the polymeric hydrogel 26 forms a layer that at least partially fills each of the plurality of depressions 22 that are defined in the substrate 15, 16 surface, where the depressions 22 have different diameters D₁, D₂ in separate, distinct regions 30C, 30D of the substrate 15, 16. In the patterned structure 17C of FIG. 1D, different polymeric hydrogels 26, 26′ having different functional group densities ρ₁, ρ₂ are positioned at distinct regions 30E, 30F across the lane 12. In the patterned structure 17D of FIG. 1E, the polymeric hydrogel 26 is positioned in each similarly-sized depression 22 defined in the substrate 15, 16.

The polymeric hydrogel(s) 26, 26′ that is/are included in each of the patterned structures 17A, 17B, 17C, 17D and the non-patterned structure may be any gel material that can swell when liquid is taken up and that can contract when liquid is removed, e.g., by drying. In an example, the polymeric hydrogel 26 includes an acrylamide copolymer, such as poly N-(5-azidoacetamidylpentyl)acrylamide-co-acrylamide, hereinafter PAZAM. When the polymeric hydrogels 26, 26′ are used, one hydrogel 26 is PAZAM, and the other hydrogel 26′ may have a higher or lower primer grafting functional group density ρ₁, ρ₂ than PAZAM. PAZAM and some other forms of the acrylamide copolymer are represented by the following structure (I):

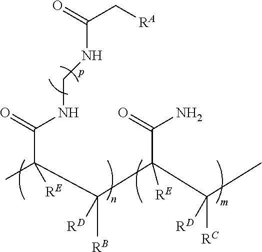

wherein:
R^{A} is selected from the group consisting of azido, optionally substituted amino, optionally substituted alkenyl, optionally substituted alkyne, halogen, optionally substituted hydrazone, optionally substituted hydrazine, carboxyl, hydroxy, optionally substituted tetrazole, optionally substituted tetrazine, nitrile oxide, nitrone, sulfate, and thiol;
R^{B} is H or optionally substituted alkyl;
R^{C}, R^{D}, and R^{E} are each independently selected from the group consisting of H and optionally substituted alkyl;
each of the —(CH₂)ₚ— can be optionally substituted;
p is an integer in the range of 1 to 50;
n is an integer in the range of 1 to 50,000; and
m is an integer in the range of 1 to 100,000.

One of ordinary skill in the art will recognize that the arrangement of the recurring “n” and “m” features in structure (I) are representative, and the monomeric subunits may be present in any order in the polymer structure (e.g., random, block, patterned, or a combination thereof).

The molecular weight of PAZAM and other forms of the acrylamide copolymer may range from about 5 kDa to about 1500 kDa or from about 10 kDa to about 1000 kDa. In a specific example, the molecular weight of the acrylamide copolymer is about 312 kDa.

In some examples, PAZAM and other forms of the acrylamide copolymer are linear polymers. In some other examples, PAZAM and other forms of the acrylamide copolymer are lightly cross-linked polymers.

In other examples, the gel material may be a variation of the structure (I).

In one example, the acrylamide unit may be replaced with N,N-dimethylacrylamide

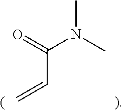

In this example, the acrylamide unit in structure (I) may be replaced with

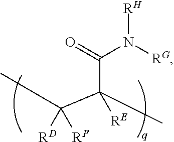

where R^{D}, R^{E}, and R^{F} are each H or a C1-C6 alkyl, and R^{G} and R^{H} are each a C1-C6 alkyl (instead of H as is the case with the acrylamide). In this example, q may be an integer in the range of 1 to 100,000. In another example, the N,N-dimethylacrylamide may be used in addition to the acrylamide unit. In this example, structure (I) may include

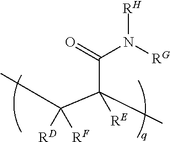

in addition to the recurring “n” and “m” features, where R^{D}, R^{E}, and R^{F} are each H or a C1-C6 alkyl, and R^{G} and R^{H} are each a C1-C6 alkyl. In this example, q may be an integer in the range of 1 to 100,000.

As another example of the polymeric hydrogel(s) 26, 26′ the recurring “n” feature in structure (I) may be replaced with a monomer including a heterocyclic azido group having structure (II):

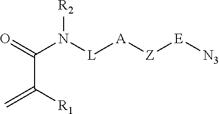

wherein R¹ is H or a C1-C6 alkyl; R₂ is H or a C1-C6 alkyl; L is a linker including a linear chain with 2 to 20 atoms selected from the group consisting of carbon, oxygen, and nitrogen and 10 optional substituents on the carbon and any nitrogen atoms in the chain; E is a linear chain including 1 to 4 atoms selected from the group consisting of carbon, oxygen and nitrogen, and optional substituents on the carbon and any nitrogen atoms in the chain; A is an N substituted amide with an H or a C1-C4 alkyl attached to the N; and Z is a nitrogen containing heterocycle. Examples of Z include 5 to 10 carbon-containing ring members present as a single cyclic structure or a fused structure. Some specific examples of Z include pyrrolidinyl, pyridinyl, or pyrimidinyl.

As still another example, the polymeric hydrogel(s) 26, 26′ may include a recurring unit of each of structure (III) and (IV):

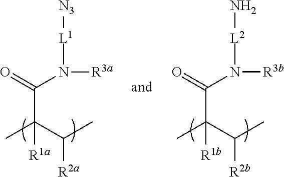

wherein each of R^{1a}, R^{2a}, R^{1b} and R^{2b} is independently selected from hydrogen, an optionally substituted alkyl or optionally substituted phenyl; each of R^{3a} and R^{3b} is independently selected from hydrogen, an optionally substituted alkyl, an optionally substituted phenyl, or an optionally substituted C7-C14 aralkyl; and each of L¹ and L² is independently selected from an optionally substituted alkylene linker or an optionally substituted heteroalkylene linker.

It is to be understood that other polymeric hydrogel(s) 26, 26′ may be used, provided that the hydrogels are suitable for grafting oligonucleotide primers 32A, 32B thereto. Some additional examples of suitable materials for the polymeric hydrogel(s) 26, 26′ include functionalized polysilanes, such as norbornene silane, azido silane, alkyne functionalized silane, amine functionalized silane, maleimide silane, or any other polysilane having functional groups that can attach the desired primer set 32A, 32B. Other examples of suitable polymeric hydrogel(s) 26, 26′ include those having a colloidal structure, such as agarose; or a polymer mesh structure, such as gelatin; or a cross-linked polymer structure, such as polyacrylamide polymers and copolymers, silane free acrylamide (SFA), or an azidolyzed version of SFA. Examples of suitable polyacrylamide polymers may be synthesized from acrylamide and an acrylic acid or an acrylic acid containing a vinyl group, or from monomers that form [2+2] photo-cycloaddition reactions. Still other examples of suitable polymeric hydrogels include mixed copolymers of acrylamides and acrylates. A variety of polymer architectures containing acrylic monomers (e.g., acrylamides, acrylates etc.) may be utilized in the examples disclosed herein, such as branched polymers, including dendrimers, and the like. For example, the monomers (e.g., acrylamide, etc.) may be incorporated, either randomly or in block, into the branches (arms) of a dendrimer.

The polymeric hydrogel(s) 26, 26′ may be formed using any suitable copolymerization process and may be deposited using any of the methods disclosed herein. For at least some of the deposition techniques, the polymeric hydrogel(s) 26, 26′ may be incorporated into a mixture, e.g., with water or with ethanol and water, prior to its application to the substrate 15, 16.

The attachment of the polymeric hydrogel(s) 26, 26′ to the single-layer substrate 15 or to the layer 20 of the multi-layer substrate 16 may be through covalent bonding. As described, in some instances, the single-layer substrate 15 or the layer 20 may first be activated, e.g., through silanization and/or plasma ashing, to facilitate the attachment of the polymeric hydrogel(s) 26, 26′ thereto. Covalent linking is helpful for maintaining the primers 32A, 32B (which are attached to the polymeric hydrogel(s) 26, 26′) in desired regions of the flow channel(s) 11 throughout the lifetime of the flow cell 10 during a variety of uses.

As mentioned, and as shown in FIG. 1B through FIG. 1E, the polymeric hydrogel 26 or 26 and 26′ has primers 32A, 32B (e.g., of a primer set) attached thereto.

As will be described in more detail in reference to the methods disclosed herein, a grafting process may be performed to graft the primers 32A, 32B to the polymeric hydrogel(s) 26, 26′ either before or after the polymeric hydrogel(s) 26, 26′ is/are deposited on the substrate 15 or 16. When the primers 32A, 32B are attached to the polymeric hydrogel(s) 26, 26′ before the hydrogel(s) 26, 26′ is/are deposited, the polymeric hydrogel(s) 26, 26′ is/are referred to as being “pre-grafted.” When the primers 32A, 32B are attached to the polymeric hydrogel(s) 26, 26′ after the hydrogel(s) 26, 26′ is/are deposited, the polymeric hydrogel(s) 26, 26′ is/are referred to as being “non-pre-grafted.”

In an example, the primers 32A, 32B may be amplification primers. In this example, the amplification primers 32A, 32B can be immobilized to the hydrogel(s) 26, 26′ by single point covalent attachment at or near the 5′ end of the primers 32A, 32B. This attachment leaves (i) an adapter-specific portion of the primers 32A, 32B free to anneal to its cognate sequencing-ready nucleic acid fragment and (ii) the 3′ hydroxyl group free for primer extension. Any suitable covalent attachment may be used for this purpose. Examples of terminated primers that may be used include alkyne terminated primers (e.g., which may attach to an azide surface moiety of the polymeric hydrogel(s) 26, 26′), or azide terminated primers (e.g., which may attach to an alkyne surface moiety of the polymeric hydrogel(s) 26, 26′), or phospho-thioate terminated primers (e.g., which may attach to a bromine surface moiety of the polymeric hydrogel(s) 26, 26′).

The primer set includes two different primers 32A, 32B that may be used in sequential paired end sequencing. As examples, the primer set may include P5 and P7 primers, P15 and P7 primers, or any combination of the PA primers, the PB primers, the PC primers, and the PD primers set forth herein. As examples, the primer set may include any combination of one PA primer and one PB, PC, or PD primer, or any combination of one PB primer and one PC or PD primer, or any combination of one PC primer and one PD primer.

The P5 primer (shown as a cleavable primer due to the cleavable nucleobase uracil, inosine, or alkene-thymidine) is:

```
P5 #1: 5′→3′
  (SEQ. ID. NO. 1)
AATGATACGGCGACCACCGAGAUCTACAC;
or
P5 #2: 5′→3′
  (SEQ. ID. NO. 2)
AATGATACGGCGACCACCGAGAnCTACAC
```

where “n” is inosine in the sequence; or

P5 #3: 5′→3′
(SEQ. ID. NO. 3)
AATGATACGGCGACCACCGAGAnCTACAC

where “n” is alkene-thymidine (i.e., alkene-dT) in the sequence.

The P7 primer (shown as cleavable primers) may be any of the following:

```
P7 #1: 5′→3′
  (SEQ. ID. NO. 4)
CAAGCAGAAGACGGCATACGAnAT;
or
P7 #2: 5′→3′
  (SEQ. ID. NO. 5)
CAAGCAGAAGACGGCATACnAGAT;
or
P7 #3: 5′→3′
  (SEQ. ID. NO. 6)
CAAGCAGAAGACGGCATACnAnAT,
```

where each instance of “n” is 8-oxoguanine.

The P15 primer (shown as a cleavable primer) is:

P15: 5′→3′
(SEQ. ID. NO. 7)
AATGATACGGCGACCACCGAGAnCTACAC

where “n” is allyl-T (a thymine nucleotide analog having an allyl functionality).

The other primers (PA-PD, shown as non-cleavable primers) mentioned above include:

```
PA 5′→3′
  (SEQ. ID. NO. 8)
GCTGGCACGTCCGAACGCTTCGTTAATCCGTTGAG
PB 5′→3′
  (SEQ. ID. NO. 9)
CGTCGTCTGCCATGGCGCTTCGGTGGATATGAACT
PC 5′→3′
  (SEQ. ID. NO. 10)
ACGGCCGCTAATATCAACGCGTCGAATCCGCAACT
PD 5′→3′
  (SEQ. ID. NO. 11)
GCCGCGTTACGTTAGCCGGACTATTCGATGCAGC
```

While not shown in the example sequences for PA-PD, it is to be understood that any of these primers may include a cleavage site, such as uracil, 8-oxoguanine, allyl-T (a thymine nucleotide analog having an allyl functionality), etc. at any point in the strand.

Each of the primers 32A, 32B disclosed herein may also include a polyT sequence at the 5′ end of the primer sequence. In some examples, the polyT region includes from 2 T bases to 20 T bases. As specific examples, the polyT region may include 3, 4, 5, 6, 7, or 10 T bases.

The 5′ end of each primer 32A, 32B may also include a linker. Any linker that includes a terminal alkyne group or another suitable terminal functional group that can attach to the surface functional groups (e.g., R^{A}) of the polymeric hydrogel 26, 26′ may be used. In one example, the primers 32A, 32B are terminated with hexynyl functional groups.

As described, primer grafting may be performed before or after the polymeric hydrogel(s) 26, 26′ is/are applied on the substrate 15, 16. In an example, grafting may involve the high-precision method described herein, flow-through deposition, dunk coating, spray coating, puddle dispensing, or by another suitable method that will attach the primer(s) 32A, 32B to the polymeric hydrogel(s) 26, 26′. Each of these example techniques may utilize a primer solution or mixture, which may include the primer(s) 32A, 32B, water, a buffer, and a catalyst. With any of the grafting methods (e.g., grafting before deposition of the polymeric hydrogel(s) 26, 26′ or after deposition of the polymeric hydrogel(s) 26, 26′), the primers 32A, 32B react with reactive groups of the polymeric hydrogel(s) 26, 26′ when the primers 32A, 32B and the polymeric hydrogel(s) 26, 26′ come into contact with one another.

In each of the patterned structures 17A, 17B, 17C, 17D and the non-patterned structure, the primers 32A, 32B that are attached to the polymeric hydrogel 26 or hydrogels 26, 26′ form distinct regions 30A, 30B, 30C, 30D, 30E, or 30F of the density gradient 28.

As shown in FIG. 1B, in the patterned structure 17A, the polymeric hydrogel 26 is deposited to form a layer with different thicknesses T₁, T₂ within respective, distinct regions 30A, 30B of the lane 12. “T₁” represent a range of thicknesses that are smaller than a range of thicknesses that are encompassed by “T₂.” The area with thicknesses T₂ has more of the hydrogel material exposed at the surface, and thus has more exposed functional groups that are able to bind to the primers 32A, 32B. Additionally, the thicker hydrogel may also mitigate surface-based interactions that can otherwise inhibit primer grafting. Thicknesses T₁, T₂ of the polymeric hydrogel 26 are varied across at least a portion of the substrate 15, 16 to define distinct regions 30A, 30B where, respectively, fewer or greater numbers of primers 32A, 32B can attach. Thus, the different thicknesses T₁, T₂ help create the primer density gradient 28. In the region 30A, the polymeric hydrogel 26 has the thicknesses T₁, which are less than the thicknesses T₂ of the polymeric hydrogel 26 in the region 30B. As described herein, the density gradient 28 includes at least two different substrate regions (i.e., regions 30A, 30B) that have a different primer density with respect to one another, and the at least two different regions 30A, 30B define the density gradient 28. In an example, the thicknesses T₁ range from about 3 nm to about 5 nm, and the thicknesses T₂ range from about 18 nm to about 20 nm. These thicknesses T₁, T₂ represent the dry form of the polymeric hydrogel 26. When wet, the thickness ranges from 50 nm (e.g., T₁) to about 200 nm (e.g., T₂). It is to be understood that dry thicknesses T₁, T₂ between 3 nm and 20 nm may be present along the width or length of the polymeric hydrogel as the thickness increases from T₁ to T₂ along the width or length.

While the patterned structure 17A is shown as including two different regions 30A, 30B, each having the polymeric hydrogel 26 applied at different thicknesses T₁, T₂, it is to be understood that any number of individual regions having different polymeric hydrogel thicknesses (e.g., T₁, T₂, etc.) may be included in the patterned structure 17A (e.g., to define the density gradient 28).

The region(s) 30B of the patterned structure 17A with the greater thicknesses T₂ will ultimately have more primers 32A, 32B attached thereto within a given area, and thus will have a higher primer density relative to other region(s) 30A that have the lesser thicknesses T₁. The region 30B with the greater thicknesses T₂ (and thus the higher primer density) can be used to bias toward the amplification of smaller DNA fragments, whereas the region 30A with the lesser thicknesses T₁ (and thus the lower primer density) can be used to bias toward the amplification of longer DNA fragments. A higher primer density makes it easier to seed an incoming DNA fragment, and thus the bias at the higher primer density areas is toward the shorter DNA fragments (which move faster than the longer DNA fragments). Similarly, a lower primer density makes it harder to seed an incoming DNA fragment, and thus the bias at the lower primer density areas is toward the longer DNA fragments (which have a better chance of reaching the primers 32A, 32B in these areas due to their length).

As shown in the patterned structure 17B of FIG. 1C, different sub-sets of depressions 22 (separated by interstitial regions 24) are included, where the different sub-sets of depressions 22 have different diameters D₁, D₂. In an example, the diameter D₁ ranges from about 630 nm to about 700 nm, and the diameter D₂ ranges from about 200 nm to about 220 nm. The depressions 22 with the larger diameter D₁ are capable of holding more primers 32A, 32B than the depressions 22 with the smaller diameter D₂. Thus, the differently sized depressions 22 and the primers 32A, 32B contained therein define distinct regions 30C, 30D of the primer density gradient 28. Each region 30C, 30D includes a plurality of the depressions with the associated diameter D₁ or D₂. As such, in this example, the substrate 15, 16 includes a plurality of depressions 22 defined therein that are separated by interstitial regions 24; the polymeric hydrogel 26 at least partially fills each of the plurality of depressions 22; and a diameter D₁, D₂ of at least some depressions 22 in the plurality of depressions 22 is varied to define distinct regions 30C, 30D of the density gradient 28 (because the differently sized depressions 22 house different numbers of primers 32A, 32B). In the region 30C, the depressions 22 have a diameter D₁, which is larger than the diameter D₂ of the depressions 22 in the region 30D. Thus, the primer density is higher in the region 30C than in the region 30D.

While the patterned structure 17B is shown as including two different regions 30C, 30D, each having different and respective depression diameters D₁, D₂, it is to be understood that any number of individual regions (e.g., 30C, 30D, etc.) having different depression diameters (e.g., D₁, D₂, etc.) may be included in the patterned structure 17B (e.g., to define the density gradient 28).

The region 30C with the bigger diameter depressions 22 (and thus the higher primer density) can be used to bias toward the amplification of longer DNA fragments, whereas the region with the smaller diameter depressions (and thus the lower primer density) can be used to bias the amplification of shorter DNA fragments. In these examples, the diameter D₁, D₂ affects the bias more than the primer density itself. The larger DNA fragments may be physically unable to enter the depressions with the smaller diameter D₂, whereas they can readily enter the larger depressions 22, D₂. While the smaller fragments may be able to enter both the larger and smaller depressions 22, the amplification bias in the larger diameters 22, D₁ is less toward the smaller fragments.

In the patterned structure 17B, many different layouts of the depressions 22 having the different diameters D₁, D₂ may be envisaged, including regular, repeating, and non-regular patterns, depending on the desired density gradient 28 that is to be defined by the regions 30C, 30D. In an example, the depressions 22 are disposed in a hexagonal grid for close packing and improved density. Other layouts may include, for example, rectilinear (rectangular) layouts, triangular layouts, and so forth. In some examples, the layout or pattern can be an x-y format in rows and columns. In other examples, the layout or pattern can be a repeating arrangement of depressions 22 and interstitial regions 24.

The layout or pattern of depressions 22 may be characterized with respect to the density (number) of the depressions 22 within a defined region 30C, 30D. For example, the depressions 22 may be present at a density of approximately 1 million per mm² within each of the regions 30C, 30D. The density may be tuned to different densities within either of the regions 30C, 30D, including, for example, a density of about 100 per mm², about 1,000 per mm², about 0.1 million per mm², about 1 million per mm², about 2 million per mm², about 5 million per mm², about 10 million per mm², about 50 million per mm², or more, or less. It is to be further understood that the density can be between one of the lower values and one of the upper values selected from the ranges above, or that other densities (outside of the given ranges) may be used. As examples, a high-density array may be characterized as having the depressions 22 separated by less than about 100 nm, a medium density array may be characterized as having the depressions 22 separated by about 400 nm to about 1 μm, and a low-density array may be characterized as having the depressions 22 separated by greater than about 1 μm.

The layout or pattern of the depressions 22 may also or alternatively be characterized in terms of the average pitch, or the spacing from the center of one depression 22 to the center of an immediately adjacent depression 22. The pattern can be regular, such that the coefficient of variation around the average pitch is small, or the pattern can be non-regular in which case the coefficient of variation can be relatively large. In either case, the average pitch can be, for example, about 50 nm, about 0.15 μm, about 0.5 μm, about 1 μm, about 5 μm, about 10 μm, about 100 μm, or more or less. The average pitch for a particular pattern of depressions 22 can be between one of the lower values and one of the upper values selected from the ranges herein. The pitch in different regions 30C, 30D may be different. Altering the pitch can also affect the bias. For example, if the depressions 22 are closer together, the larger and slower fragments have a higher chance of becoming seeded and amplifying.

The size of each depression 22 in the patterned structure 17B may be characterized by its volume, opening area, and/or depth. For example, the volume can range from about 1×10⁻³ μm³ to about 100 μm³, e.g., about 1×10⁻² μm³, about 0.1 μm³, about 1 μm³, about 10 μm³, or more, or less. For another example, the opening area can range from about 1×10⁻³ μm² to about 100 μm², e.g., about 1×10⁻² μm², about 0.1 μm², about 1 μm², at least about 10 μm², or more, or less. For still another example, the depth can range from about 0.1 μm to about 100 μm, e.g., about 0.5 μm, about 1 μm, about 10 μm, or more, or less. For another example, the depth can range from about 0.1 μm to about 100 μm, e.g., about 0.5 μm, about 1 μm, about 10 μm, or more, or less.

Turning now to FIG. 1D, in the patterned structure 17C, two polymeric hydrogels 26, 26′ with different functional group densities ρ₁, ρ₂ are applied within the lane 12. The different functional group densities ρ₁, ρ₂ enable the polymeric hydrogels 26, 26′ to graft different numbers of primers, thus creating the regions 30E, 30F of the primer density gradient 28. In the region 30E, the polymeric hydrogel 26 has a first functional group density ρ₁, which is less than the functional group density ρ₂ of the polymeric hydrogel 26′ within the region 30F. In this example, two different polymeric hydrogels 26, 26′ with different densities of primer grafting functional groups are utilized.

While the patterned structure 17C is shown as including two different regions 30E, 30F, each having different and respective functional group density ρ₁, ρ₂, it is to be understood that any number of individual regions having different functional group densities (e.g., ρ₁, ρ₂, etc.) may be included in the patterned structure 17C (e.g., to define the density gradient 28).

The region 30E or 30F of the patterned structure 17C with the greater functional group density ρ₁ or ρ₂ will ultimately have more primers 32A, 32B attached thereto within a given area (relative to the other region 30F or 30E). In the example shown in FIG. 1D, the region 30F with the greater functional group density ρ₂ (and thus the higher primer density) can be used to bias toward the amplification of shorter DNA fragments, whereas the region 30E with the lesser functional group density ρ₁ (and thus the lower primer density) can be used to bias toward the amplification of longer DNA fragments.

In another example that can be represented by FIG. 1D, the two polymeric hydrogels 26, 26′ have different porosities. The porosities enable the polymeric hydrogels 26, 26′ to graft different numbers of primers, thus creating the regions 30E, 30F of the primer density gradient 28. As an example, in the region 30E, the polymeric hydrogel 26 may have a first porosity, which is less than the porosity of the polymeric hydrogel 26′ within the region 30F. The pore size (diameter) in the regions 30E, 30F range from about 1 nm to about 1 μm, as long as the pore size varies in the regions 30E, 30F to create the gradient. In another example, the pore size across the regions 30E, 30F ranges from about 1 nm to about 100 nm. The polymeric hydrogel 26′ with the higher porosity has a greater number of small pores or a more intricate pore structure than the hydrogel 26, which provides more surface area per unit volume for primer 32A, 32B attachment.

While the different porosities are described as making up two different regions 30E, 30F, it is to be understood that any number of individual regions having different porosities may be included to define the density gradient 28.

The region(s) 30F with the greater porosity will ultimately have more primers 32A, 32B attached thereto within a given area, and thus will have a higher primer density relative to other region(s) 30E that have the lesser porosity. The region 30F with the greater porosity (and thus the higher primer density) can be used to bias toward the amplification of smaller DNA fragments, whereas the region 30E with the lesser porosity (and thus the lower primer density) can be used to bias toward the amplification of longer DNA fragments. The porosity can also impact the accessibility of enzyme diffusion into the area, thus furthering the bias toward amplification of the smaller DNA fragment in highly porous regions.

While not shown in FIG. 1D, the architecture may alternatively include a single polymeric hydrogel 26 in the lane 12, as opposed to multiple hydrogels 26, 26′ with different functional group densities ρ₁, ρ₂. In this example, the primer density gradient 28 can be achieved across a length or width of the lane 12 by controlling the temperature and/or reaction kinetics at particular areas during grafting of the primers 32A, 32B (also referred to as primer lawn grafting). For example, a portion of the substrate 15, 16 may be heated during primer grafting, which will speed up grafting and generate the region 30F with a higher primer density. Similarly, another portion of the substrate 15, 16 may be cooled or exposed to a chilled reagent during primer grafting, which will slow down grafting and generate the region 30E with a lower primer density.

The patterned substrates 17A, 17B, 17C may also be designed so that the higher primer density regions, e.g., 30B, 30C, 30F are positioned adjacent to an inlet of the flow cell 10 so that the shorter and faster DNA library fragments quickly seed in these regions, e.g., 30B, 30C, 30F, leaving the slower and longer DNA library fragments available for seeding in the lower primer density regions 30A, 30D, and 30E.

While the flow cells 10 described in reference to FIG. 1B through FIG. 1D each include the density gradient, it is to be understood that some of the methods described herein can be used with flow cells that do not include the primer density gradient.

One example of this flow cell 10 is similar to the structure 17C shown in FIG. 1D, except that the primers 32A, 32B are relatively uniformly distributed across a single polymeric hydrogel 26 in the lane 12.

Another example of this flow cell 10 includes the patterned structure 17D shown in FIG. 1E. In this example structure 17D, the depressions 22 have the same size, and thus the primer density is relatively consistent across the flow cell 10.

Methods for preparing the various patterned structures 17A, 17B, 17C, 17D, and for preparing the non-patterned structure as part of a process of forming various examples of the flow cell 10, will now be described.

### Flow Cell Preparation Methods

The methods for preparing the patterned structures 17A, 17B, 17C with the primer density gradient generally involve depositing a polymeric hydrogel 26 over at least a portion of a substrate 15, 16; and attaching a plurality of oligonucleotide primers 32A, 32B to at least a portion of the polymeric hydrogel 26, such that the plurality of oligonucleotide primers 32A, 32B forms a density gradient 28 across the substrate 15, 16.

The methods for preparing the patterned structure 17D without the primer density gradient generally involve depositing a polymeric hydrogel 26 within each depression 22 defined in a substrate 15, 16; and attaching a plurality of oligonucleotide primers 32A, 32B to the polymeric hydrogel 26 in each of the depressions 22.

The methods for preparing the non-patterned structure without the primer density gradient generally involve depositing a polymeric hydrogel 26 within a lane 12 defined in a substrate 15, 16; and attaching a plurality of oligonucleotide primers 32A, 32B to the polymeric hydrogel 26 in the lane 12.

Specific examples of the various methods will now be described.

### Method of Preparing the Patterned Structure 17A

An example method for forming the patterned structure 17A in shown in FIG. 2A through FIG. 2D. In this example method, the depositing of the polymeric hydrogel 26 is controlled to generate a polymeric hydrogel thickness that is varied across at least a portion of the substrate 15, 16; and the attaching of the plurality of oligonucleotide primers 32A, 32B to the at least the portion of the polymeric hydrogel 26 defines distinct regions 30A, 30B of the density gradient 28 within the at least the portion of the substrate 15, 16.

FIG. 2A shows a structure that includes the single-layer substrate 15 or the multi-layer substrate 16 (including the base support 18 and the additional layer 20 thereon) with the lane 12 defined therein. The substrate 15, 16 may include any suitable materials disclosed herein.

The lane 12 may be etched, nanoimprinted, embossed, or otherwise defined in the surface of the substrate 15 or 16. As an example, when the single-layer substrate 15 or layer 20 includes a resin, a working stamp (not shown) including a negative replica of the lane 12 may be pressed into the resin material while the resin is soft. Curing and/or drying of the resin may then be performed via exposure to actinic radiation or heat, either before or after the working stamp is removed. Removal of the working stamp forms the lane 12.

In some instances, the structure shown in FIG. 2A may be activated, e.g., by silanizing the substrate 15, 16 and/or by plasma ashing the substrate 15, 16.

In examples, activation of the single-layer substrate 15 or of the layer 20 of the multi-layer substrate 16 involves exposing the single-layer substrate 15 or the layer 20 to a suitable silane in an appropriate solvent (referred to herein as “silanization” of the single-layer substrate 15 or the layer 20 of the multi-layer substrate 16). Silanization involves the application of a silane or silane derivative over the surface of the single-layer substrate 15 or of the layer 20. Some example silane derivatives include a cycloalkene unsaturated moiety, such as norbornene, a norbornene derivative (e.g., a (hetero)norbornene including an oxygen or nitrogen in place of one of the carbon atoms), transcyclooctene, transcyclooctene derivatives, transcyclopentene, transcycloheptene, trans-cyclononene, bicyclo[3.3.1]non-1-ene, bicyclo[4.3.1]dec-1 (9)-ene, bicyclo[4.2.1]non-1(8)-ene, and bicyclo[4.2.1]non-1-ene. Any of these cycloalkenes can be substituted, for example, with an R group, such as hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl. An example of the norbornene derivative includes [(5-bicyclo[2.2.1]hept-2-enyl)ethyl]trimethoxysilane. Other example silane derivatives include a cycloalkyne unsaturated moiety, such as cyclooctyne, a cyclooctyne derivative, or bicyclononynes (e.g., bicyclo[6.1.0]non-4-yne or derivatives thereof, bicyclo[6.1.0]non-2-yne, or bicyclo[6.1.0]non-3-yne). These cycloalkynes can be substituted with any of the R groups described herein. The method used to apply the silane or silane derivative may vary depending upon the silane or silane derivative that is being used. Examples of suitable silanization methods include vapor deposition (e.g., a YES method), spin coating, or other deposition methods.

In another example, activation of the single-layer substrate 15 or of the layer 20 involves plasma ashing the single-layer substrate 15 or the layer 20. Plasma ashing involves the generation of —OH groups at a surface via exposure of the surface to oxygen plasma.

As shown in FIG. 2B, the polymeric hydrogel 26 may then be deposited over the single-layer substrate 15 or over the layer 20 of the multi-layer substrate 16. The polymeric hydrogel 26 may include any suitable example described herein and may be applied/deposited over the substrate 15, 16 using any suitable deposition technique disclosed herein. The deposition of the polymeric hydrogel 26 forms a layer of the polymeric hydrogel 26 (i) having a first thickness T₁ within a first region 30A of the substrate 15, 16 and (ii) having a second thickness T₂ within a second region 30B of the substrate 15, 16, where the two thicknesses T₁, T₂ are different with respect to one another. In examples, the thicknesses T₁, T₂ of the polymeric hydrogel 26 layer within the respective regions 30A, 30B are generated by controlling the deposition of the polymeric hydrogel 26. The thickness gradient may also be generated through thermal profiles when the polymeric hydrogel 26 is deposited. By changing the temperature of the substrate 15, 16 as the polymeric hydrogel 26 is deposited will change the local surface viscosity. The thermal profiles can be used to reduce the viscosity of the hydrogel 26 in some region(s), which leads to a thinner hydrogel 26, and to increase the viscosity of the hydrogel 26 in other region(s), which leads to a thicker hydrogel 26. In another example, a hydrogel—with a lower temperature than the substrate 15, 16 to which it is introduced—is deposited at a flow rate that creates a thickness gradient as the hydrogel 26 cools. In still another example, the thickness gradient can be created using a charge gradient. The charge gradient can be generated during ashing and dry down techniques, which creates a thinner hydrogel at one end of the substrate 15, 16 by blowing air at a suitable Reynolds number. For example, a precision gantry tool may be used to deposit a first layer of the polymeric hydrogel 26 in the lane 12, and then used to deposit additional material only in a portion of the lane 12 to add to the thickness in that portion. In FIG. 2B, the thickness T₂ (in the region 30B) is shown as being greater than the thickness T₁ (in the region 30A), and the thickness T₂ gradually increase across the region 30B away from the region 30A. The slope of the polymeric hydrogel 26 in the region 30B provides a greater surface area for primer attachment than the slope of the polymeric hydrogel 26 in the region 30A. The different thicknesses T₁, T₂ at the respective regions 30A, 30B may be any suitable thicknesses or ranges of thicknesses disclosed herein. The two regions 30A, 30B will ultimately define distinct portions of the density gradient 28. It is to be understood, however, that any number of individual regions (30A, 30B, etc.) may be included, where each region has its own respective polymeric hydrogel 26 thickness (or range of thicknesses), and where the regions will ultimately and collectively define the density gradient 28.

While not shown in FIG. 2B, in some instances, the polymeric hydrogel 26 is a pre-grafted polymeric hydrogel 26 (and thus the polymeric hydrogel 26 has primers 32A, 32B attached thereto prior to deposition of the polymeric hydrogel 26 over the substrate 15, 16). In these instances, the deposition of the pre-grafted polymeric hydrogel 26 forms the regions (e.g., 30A, 30B, etc.) that define the density gradient 28, and the density gradient 28 is formed contemporaneously with the deposition of the polymeric hydrogel 26 (and the primers 32A, 32B attached thereto). At least two distinct regions 30A, 30B of the density gradient 28 are defined, wherein each of the distinct regions 30A, 30B has a different primer density. For example, the portion of the polymeric hydrogel 26 within the region 30B (i.e., the portion of the polymeric hydrogel 26 having the thickness T₂) will have more primers 32A, 32B attached thereto, relative to the portion of the polymeric hydrogel 26 within the region 30A (i.e., the portion of the polymeric hydrogel 26 having the thickness T₁). As such, the primer density will ultimately be greater in the region 30B (which has the polymeric hydrogel 26 thickness T₂), relative to the primer density in the region 30A (which has the polymeric hydrogel 26 thickness T₁). This difference in primer density (between any number of discrete regions 30A, 30B, etc.) defines the density gradient 28 (see FIG. 2D).

In other instances, the polymeric hydrogel 26 is not pre-grafted with the primers 32A, 32B, and the density gradient 28 is formed later in the method (see FIG. 2D).

After the polymeric hydrogel 26 (pre-grafted or non-pre-grafted) has been applied over the single-layer substrate 15 or over the layer 20 of the multi-layer substrate 16, a polishing process may be performed, which generates the structure shown in FIG. 2C. During the polishing process, portions of the polymeric hydrogel 26 on the bonding regions 34 are removed, while the polymeric hydrogel 26 (i.e., having the thickness T₁, T₂) within the lane 12 remains intact.

The polishing process may be performed with a chemical slurry (including, e.g., an abrasive, a buffer, a chelating agent, a surfactant, and/or a dispersant) that can remove the polymeric hydrogel 26, e.g., from bonding regions 34 of the substrate 15, 16, without deleteriously affecting the underlying substrate 15, 16 and without affecting the polymeric hydrogel 26 layer within the lane 12. Polishing may also be performed with a solution that does not include the abrasive particles. The polishing process may also be performed using (a) polishing head(s)/pad(s) or (an)other polishing tool(s). As an example, the polishing head may be a Strasbaugh ViPRR II polishing head.

As shown in FIG. 2D, in some examples (such as those in which the non-pre-grafted polymeric hydrogel 26 is used), the method proceeds by grafting the plurality of oligonucleotide primers 32A, 32B to the polymeric hydrogel 26. Grafting may be performed using any suitable technique disclosed herein. In these examples, the attachment of the primers 32A, 32B to the polymeric hydrogel 26 having the different thicknesses T₁, T₂ within the regions 30A, 30B generates the density gradient 28. It is to be understood that the density of the primers 32A, 32B differs within the respective regions 30A, 30B to define the density gradient 28. Thus, in examples, at least two distinct regions 30A, 30B of the density gradient 28 are defined, and each of the distinct regions 30A, 30B has a different primer density. The portion of the polymeric hydrogel 26 within the region 30B (i.e., having the thickness T₂) will graft more primers 32A, 32B thereto due to the increased exposed surface area, relative to the portion of the polymeric hydrogel 26 within the region 30A (i.e., having the thickness T₁). As such, the primer density is greater in the region 30B (which has the polymeric hydrogel 26 thickness T₂), relative to the primer density in the region 30A (which has the polymeric hydrogel 26 thickness T₁). This difference in primer density (between any number of discrete regions 30A, 30B, etc.) defines the density gradient 28.

In this example method, the formation of the density gradient 28 can be further controlled using temperature and/or the reaction kinetics of primer grafting. As such, in some examples, during the attaching of the plurality of oligonucleotide primers 32A, 32B to the at least the portion of the polymeric hydrogel 26 (e.g., the portions that form the region 30A or 30B), the method further comprises controlling a temperature of the substrate 15, 16 to facilitate formation of the density gradient 28. To increase the primer grafting at a portion of the polymeric hydrogel 26, the portion may be heated during primer grafting, which will speed up grafting at the portion. Similarly, to decrease primer grafting at another portion of the polymeric hydrogel 26, the other portion may be cooled or exposed to a chilled reagent during primer grafting, which will slow down grafting.

In still further examples, during the attaching of the plurality of oligonucleotide primers 32A, 32B to the at least the portion of the polymeric hydrogel 26 (e.g., the portions that form the regions 30A or 30B), the method further comprises controlling a flow rate of a primer mixture including the plurality of oligonucleotide primers to facilitate formation of the density gradient 28. A slower flow rate allows more time for mass transfer and reaction kinetics, and thus more primer grafting.

In yet further examples, during the attaching of the plurality of oligonucleotide primers 32A, 32B to the at least the portion of the polymeric hydrogel 26, the method further comprises controlling a concentration of a primer mixture (including the plurality of oligonucleotide primers 32A, 32B) to facilitate formation of the density gradient.

Additionally, any combination of temperature, flow rate, and primer concentration may be used to adjust the bias during the attachment of the oligonucleotide primers 32A, 32B.

The patterned structure 17A can be used as an open-wafer substrate. Alternatively, the lid or a second patterned structure 17A may be bonded to the patterned structure 17A at the bonding regions. The patterned structure 17A and the lid or the second patterned structure 17A may be bonded using any suitable technique, such as laser bonding, diffusion bonding, anodic bonding, eutectic bonding, plasma activation bonding, glass frit bonding, or others methods known in the art.

### Method of Preparing the Patterned Structure 17B

An example method for forming the patterned structure 17B i shown in FIG. 3A through FIG. 3D. In this example method, prior to depositing the polymeric hydrogel 26, the method further comprises patterning the substrate 15, 16 to include a plurality of depressions 22 that are separated by interstitial regions 24, wherein a diameter of at least some depressions 22 in the plurality of depressions 22 is varied to define distinct regions 30C, 30D of the density gradient 28.

FIG. 3A shows a structure that includes the single-layer substrate 15 or the multi-layer substrate 16 (including the base support 18 and the additional layer 20 thereon). The substrate 15, 16 may include any suitable materials disclosed herein. As shown in the figure, the substrate 15, 16 includes the plurality of depressions 22 defined therein, where the depressions 22 are separated by interstitial regions 24. As further shown in the figure, the depressions 22 within the region 30C of the substrate 15, 16 have a first diameter D₁, and the depressions 22 within the separate region 30D have a second diameter D₂, which is smaller than the first diameter D₁. The different regions 30C, 30D will ultimately define individual portions of the density gradient 28.

The depressions 22 having the first and second diameter D₁, D₂ may be defined in the single-layer substrate 15 or in the layer 20 of the multi-layer substrate 16 using any suitable patterning technique disclosed herein, such as nanoimprint lithography, photolithography, micro-etching techniques, etc. As an example, when the single-layer substrate 15 or layer 20 includes a resin, a working stamp (not shown) including a negative replica of the depressions 22 (i.e., a negative replica of depressions 22 having the first diameter D₁ and having the second diameter D₂) may be pressed into the resin material while the resin is soft. Curing and/or drying of the resin may then be performed via exposure to actinic radiation or heat, either before or after the working stamp is removed. Removal of the working stamp forms the depressions 22 (having the different diameters D₁, D₂) and the interstitial regions 24 that are shown in FIG. 3A.

In some instances, the structure shown in FIG. 3A may be activated, e.g., by silanizing the substrate 15, 16 and/or by plasma-ashing the substrate 15, 16 (similar to the processes described herein in reference to the activation of the structure shown in FIG. 2A).

As shown in FIG. 3B, the polymeric hydrogel 26 may then be deposited over the single-layer substrate 15 or over the layer 20 of the multi-layer substrate 16, such that the polymeric hydrogel 26 at least partially fills each of the plurality of depressions 22 (i.e., the depressions 22 in the region 30C having the first diameter D₁ and the depressions 22 in the region 30D having the second diameter D₂) and such that the polymeric hydrogel 26 overlies the interstitial regions 24. The polymeric hydrogel 26 may include any suitable example disclosed herein and may be applied/deposited over the single-layer substrate 15 or layer 20 using any suitable deposition technique disclosed herein.

While not shown in FIG. 3B, in some instances, the polymeric hydrogel 26 that is deposited is a pre-grafted polymeric hydrogel 26 (and thus the polymeric hydrogel 26 has primers 32A, 32B attached thereto prior to deposition of the polymeric hydrogel 26 over the substrate 15, 16). In these instances, the deposition of the pre-grafted polymeric hydrogel 26 forms the density gradient 28. In some of these instances, two distinct regions 30C, 30D of the density gradient 28 are defined, wherein each of the distinct regions 30C, 30D has a different primer density. For example, the depressions 22 within the region 30C (having first diameter D₁) will house more primers 32A, 32B than the depressions 22 within the region 30D (having the second diameter D₂). As such, the primer density will ultimately be greater in the region 30C (which has the depressions 22 with the first diameter D₁ defined therein), relative to the primer density in the region 30D (which has the depressions 22 with the second diameter D₂ defined therein). This difference in primer density (between any number of discrete regions 30C, 30D, etc.) defines the density gradient 28.

After the (pre-grafted or non-pre-grafted) polymeric hydrogel 26 has been applied over the single-layer substrate 15 or over the layer 20 of the multi-layer substrate 16, a polishing process may be performed, which generates the structure shown in FIG. 3C. The polishing process may be performed as described herein in reference to FIG. 2C.

Referring now to FIG. 3D, in some examples (i.e., when the non-pre-grafted polymeric hydrogel 26 is used), the method proceeds by grafting the plurality of oligonucleotide primers 32A, 32B to the polymeric hydrogel 26 in each of the depressions 22. Grafting may be performed using any suitable technique disclosed herein. When the non-pre-grafted polymeric hydrogel 26 is used, the attachment of the primers 32A, 32B to the polymeric hydrogel 26 within the depressions 22 (i.e., the depressions 22 having the different diameters D₁, D₂) forms the regions 30C, 30D and the density gradient 28.

More polymeric hydrogel 26 is present in the depressions 22 with the larger diameter D₁ than in the depressions 22 with the smaller diameter D₂. Thus, there are more primer binding functional groups available in the depressions 22, D₁ than the depressions 22, D₂. Thus, the polymeric hydrogel 26 within the depressions 22 having the larger diameter (e.g., D₁) will ultimately graft more primers 32A, 32B (which creates region 30C) relative to the polymeric hydrogel 26 within the depressions 22 having the smaller diameter (e.g., D₂) (which creates region 30D). As such, the primer density is greater in the region 30C than the primer density in the region 30D. This difference in primer density (between any number of discrete regions 30C, 30D, etc.) forms the density gradient 28.

In examples, during the attaching of the plurality of oligonucleotide primers 32A, 32B to the at least the portion of the polymeric hydrogel 26 (i.e., the portions that form the region 30C or 30D), the method further comprises controlling a temperature of the substrate 15, 16 to enhance formation of the density gradient 28. To increase the primer grafting in the depressions 22 with the greater diameter D₁, these depressions 22, D₁ may be heated during primer grafting, which will speed up grafting. Similarly, to decrease primer grafting in the depressions 22 with the smaller diameter D₂, these depressions 22, D₂ may be cooled or exposed to a chilled reagent during primer grafting, which will slow down grafting. In further examples, during the attaching of the plurality of oligonucleotide primers to the hydrogel 26 in the depressions 22 (with D₁ or D₂), the method further comprises controlling a flow rate of a primer mixture including the plurality of oligonucleotide primers to enhance formation of the density gradient.

The patterned structure 17B can be used as an open-wafer substrate. Alternatively, the lid or second patterned structure 17B maybe bonded to the patterned structure 17B at the bonding regions 34. The patterned structure 17B and the lid or the second patterned structure 17B may be bonded using any suitable technique described herein.

The method described for preparation of the patterned structure 17B may also be used to prepare the patterned structure 17D, except that the depressions 22 that are formed have the same diameter and the primer grafting is relatively uniform across the depressions 22.

### Method of Preparing Patterned Structure 17C

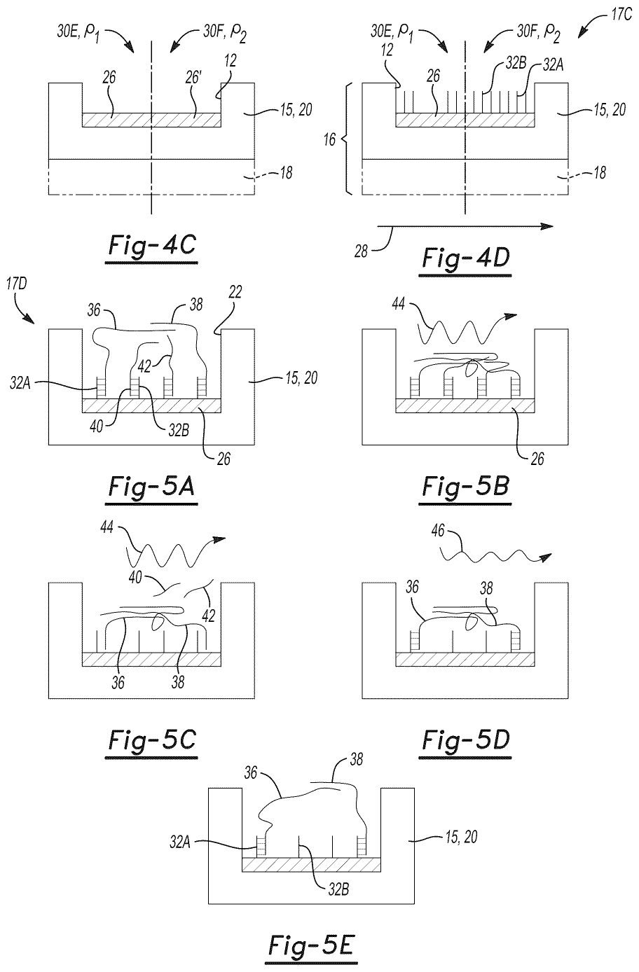

An example method for forming the patterned structure 17C is shown in FIG. 4A through FIG. 4D. In this example method, a functional group density ρ₁, ρ₂ or porosity is varied across at least a portion of the substrate 15, 16 to define distinct regions 30E, 30F of the density gradient 28.

FIG. 4A shows a structure that includes the single-layer substrate 15 or the multi-layer substrate 16 (including the base support 18 and the additional layer 20 thereon) with the lane 12 defined therein. The substrate 15, 16 may include any suitable materials disclosed herein. The lane 12 may be formed as described in reference to FIG. 2A.

The structure shown in FIG. 4A may be activated, e.g., by silanizing the substrate 15, 16 and/or by plasma ashing the substrate 15, 16. Activation of the structure shown in FIG. 4A may be performed in a manner similar to that described in reference to the activation of the structure of FIG. 2A.

As shown in FIG. 4B, the polymeric hydrogels 26, 26′ (with different functional group densities ρ₁, ρ₂ or different porosities) may then be deposited over respective portions of the single-layer substrate 15 or over the layer 20 of the multi-layer substrate 16. The polymeric hydrogels 26, 26′ may be any suitable example disclosed herein, as long as the density of the primer grafting functional groups or the porosity is different. In the illustrated example, the two functional group densities ρ₁, ρ₂ are different with respect to one another, and ρ₂ is greater than ρ₁. While two hydrogels 26, 26′ are depicted, it is to be understood that any number of individual regions (30E, 30F, etc.) may be included, where each region has its own respective functional group density (or range of functional group densities), and where the regions will ultimately and collectively define the density gradient 28.

Both primer grafting functional groups and porosity are a function of the hydrogel material that is used. Different hydrogels will have different numbers of primer grafting functional groups and will create different porosity. The porosity can also be altered by using different concentrations during deposition, different molecular weights, and/or different casting temperatures.

The respective hydrogels 26, 26′ may be deposited using selective deposition techniques (e.g., inkjet or microcontact printing). Alternatively, one portion of the lane 12 may be covered (e.g., via a photoresist or other suitable mask) while one hydrogel 26 is deposited, and then the photoresist or mask is removed and the other of the hydrogels 26′ is deposited. The hydrogel 26′ may be deposited using any suitable technique under high ionic strength conditions, e.g., in the presence of 10×PBS, NaCl, KCl, etc. When the deposition of the hydrogel 26′ is performed under high ionic strength, the hydrogel 26′ does not deposit on or adhere to the polymeric hydrogel 26.

While not shown in FIG. 4B, in some instances, the polymeric hydrogels 26, 26′ are pre-grafted polymeric hydrogels 26, 26′, and thus each of the polymeric hydrogels 26, 26′ has primers 32A, 32B attached thereto prior to their respective depositions over the designated portions of the lane 12. In these instances, the deposition of the pre-grafted polymeric hydrogel 26 forms the regions (e.g., 30E, 30F, etc.) that define the density gradient 28. In the example shown, two distinct regions 30E, 30F of the density gradient 28 are defined, wherein each of the distinct regions 30E, 30F has a different primer density or different porosity. Any number of regions may be formed using different hydrogels 26, 26′ with different functional groups densities ρ₁, ρ₂ or porosities.

The polymeric hydrogel 26 having the smaller functional group density ρ₁ or porosity will have fewer primers 32A, 32B attached thereto and will form the region 30E, and the polymeric hydrogel 26′ having the greater functional group density ρ₂ or porosity will have more primers 32A, 32B attached thereto and will form the region 30F. As such, the primer density will ultimately be greater in the region 30F (which has the functional group density ρ₂ or porosity), relative to the primer density in the region 30E (which has the functional group density ρ₁ or porosity). This difference in primer density or porosity (between any number of discrete regions 30E, 30F, etc.) forms the density gradient 28.

After the polymeric hydrogel 26, 26′ (pre-grafted or non-pre-grafted) has been applied over the single-layer substrate 15 or over the layer 20 of the multi-layer substrate 16, a polishing process may be performed, which generates the structure shown in FIG. 4C. The polishing process may be performed as described herein.

Referring now to FIG. 4D, in examples where the non-pre-grafted polymeric hydrogel 26 is used, the method proceeds by grafting the plurality of oligonucleotide primers 32A, 32B to the polymeric hydrogels 26, 26′. Grafting may be performed using any suitable technique disclosed herein. The attachment of the primers 32A, 32B to the polymeric hydrogels 26, 26′ forms the regions 30E, 30F that generate the density gradient 28. As described, the density of the primers 32A, 32B differs within the respective regions 30E, 30F to define the gradient 28. Thus, in examples, at least two distinct regions 30E, 30F of the density gradient 28 are defined, and each of the distinct regions 30E, 30F has a different primer density. This difference in primer density (between any number of discrete regions 30E, 30F, etc.) forms the density gradient 28.

In examples, during the attaching of the plurality of oligonucleotide primers 32A, 32B to the at least the portion of the polymeric hydrogels 26, 26′, the method further comprises controlling a temperature of the substrate 15, 16 to enhance formation of the density gradient 28. To increase the primer grafting to the polymeric hydrogel 26′, the polymeric hydrogel 26′ may be heated during primer grafting, which will speed up grafting. Similarly, to decrease primer grafting to the polymeric hydrogel 26, the polymeric hydrogel 26 may be cooled or exposed to a chilled reagent during primer grafting, which will slow down grafting. In further examples, during the attaching of the plurality of oligonucleotide primers to the hydrogels 26, 26′ the method further comprises controlling a flow rate of a primer mixture including the plurality of oligonucleotide primers to enhance formation of the density gradient.

The patterned structure 17C can be used as an open-wafer substrate. Alternatively, the lid or second patterned structure 17C maybe bonded to the patterned structure 17C at the bonding regions 34. The patterned structure 17C and the lid or the second patterned structure 17C may be bonded using any suitable technique described herein.

It is to be understood that any of the example methods disclosed herein in reference to the FIG. 2 through FIG. 4 series may be combined and/or modified to generate a structure having any desired features of the patterned structures 17A, 17B, 17C disclosed herein. As one example, a flow cell 10 may include the hydrogels 26, 26′ with different functional group densities ρ₁, ρ₂ and they hydrogels may be formed with the thicknesses T₁, T₂.

### Method of Preparing Non-Patterned Structure

The non-patterned structure can be formed by defining the lane 12 in the substrate 15, 16; depositing the polymeric hydrogel 26 in the lane (e.g., via blanket deposition and polishing from the binding regions 34); and grafting primers 32A, 32B across the polymeric hydrogel 26. These processes may be performed via the various processes described herein.

The non-patterned structure can be used as an open-wafer substrate. Alternatively, the lid or a non-patterned structure may be bonded to the patterned structure at the bonding regions. The non-patterned structure and the lid or the second non-patterned structure may be bonded using any suitable technique set forth herein.

### Methods of Biasing DNA Size for Amplification

Any of the flow cells 10 described herein can be used in a method that enables biasing of the library fragments that are amplified. The patterned structures 17A through 17C alone can enable the bias as described in reference to the various structures. In these examples, the method generally includes generating library templates, introducing the library templates to the flow cell 10 including the patterned structure 17A, 17B, or 17C, and initiating amplification of the library templates.

At the outset of template strand formation, the library templates may be prepared from any nucleic acid sample (e.g., a DNA sample or an RNA sample). The DNA nucleic acid sample may be fragmented into single-stranded, similarly sized (e.g., <1000 bp) DNA fragments. The RNA nucleic acid sample may be used to synthesize complementary DNA (cDNA), and the cDNA may be fragmented into single-stranded, similarly sized (e.g., <1000 bp) cDNA fragments. During preparation, adapters may be added to the ends of any of the fragments. Through reduced cycle amplification, different motifs may be introduced in the adapters, such as sequencing primer binding sites, indices, and regions that are complementary to the primers 32A, 32B on the patterned structures 17A, 17B, 17C. In some examples, the fragments from a single nucleic acid sample have the same adapters added thereto. The final library templates include the DNA or cDNA fragment and adapters at both ends. The DNA or cDNA fragment represents the portion of the final library template that is to be sequenced.

When the library templates are introduced into the flow cell 10 including the patterned structure 17A, 17B, or 17C, the shorter DNA library fragments are more likely to seed in the higher primer density regions 30B, 30C, 30F, leaving the longer DNA library fragments to seed in the lower primer density regions 30A, 30D, 30E.

Amplification of the seeded template nucleic acid strand(s) may be initiated to form respective clusters of the seeded template strands across the patterned structures 17A (in the lane 12), 17B (in the depressions 12), 17C (in the lane 12). In this example, an amplification mix is added to the flow cell 10. The amplification mix includes nucleotides, a polymerase, and accessory proteins. The amplification mix may also include a buffer agent (e.g., Tris), enzymes, stabilizers, a metal co-factor, a surfactant (e.g., TWEEN polysorbates), and/or a co-solvent (e.g., glycerol, dimethylformamide, etc.). When amplification is initiated, the seeded library templates are copied from the hybridized primers by 3′ extension using a high-fidelity DNA polymerase. The original library templates are denatured, leaving the copies immobilized all around the patterned structures 17A (in the lane 12), 17B (in the depressions 12), 17C (in the lane 12). Isothermal bridge amplification or some other form of amplification may be used to amplify the immobilized copies. For example, the copied templates loop over to hybridize to an adjacent, complementary primer 32A or 32B, and a polymerase copies the copied templates to form double stranded bridges, which are denatured to form two single stranded strands. These two strands loop over and hybridize to adjacent, complementary primers 32A or 32B and are extended again to form two new double stranded loops. The process is repeated on each template copy by cycles of isothermal denaturation and amplification to create dense clonal clusters. Each cluster of double stranded bridges is denatured. In an example, the reverse strand is removed by cleaving at the cleavage site (e.g., specific base cleavage), leaving forward template strands. In another example, the forward strand is removed by cleaving at the cleavage site, leaving reverse template strands. This example of clustering is referred to as bridge amplification, and is one example of the amplification that may be performed. It is to be understood that other amplification techniques may be used, e.g., exclusion amplification, where seeding and amplification occur almost simultaneously.

Alternatively, the method itself can enable the bias. These methods can be used in conjunction with the structures 17A through 17C to enhance the bias that the structure enables, or can be used with the patterned structure 17D or non-patterned structure that do not contribute to the bias.

In these examples, the method generally includes selecting a target fragment length to be amplified using adapter-tagged DNA fragments; introducing the adapter-tagged DNA fragments to a flow cell 10 including primers 32A, 32B; and based on the selection of the target fragment length, one of: (i) initiating sequential seeding and amplification of the adapter-tagged DNA fragments to shift a bias toward shorter fragments of the adapter-tagged DNA fragments; or (ii) initiating concurrent seeding and amplification of the adapter-tagged DNA fragments to shift the bias toward longer fragments of the adapter-tagged DNA fragments.

The bias can be further shifted by adjusting a concentration of the shorter fragments or the longer fragments that is introduced into the flow cell 10.

To initiate sequential seeding and amplification, the library fragments may be introduced into the flow cell 10 for seeding, followed by the amplification mix described herein. Both shorter and longer fragments will seed in this example. However, amplification of the shorter and longer fragments occurs simultaneously, and the shorter fragments amplifying faster. Thus, this technique biases toward amplification of the shorter library fragments because once amplification is initiated, they will multiple faster than any seeded longer library fragments. This example method may further include shifting the bias more toward the larger fragments by lowering a temperature during sequential seeding and amplification.

To initiating concurrent seeding and amplification, the library fragments are introduced into the flow cell 10 with an extension amplification mix. An example of the extension amplification mix includes nucleotides, a recombinase, a polymerase, and accessory proteins. The extension amplification mix may also include a buffer agent (e.g., Tris), enzymes, stabilizers, a metal co-factor, a surfactant (e.g., TWEEN polysorbates), and/or a co-solvent (e.g., glycerol, dimethylformamide, etc.). The ExAMP reagents available from Illumina, Inc. are examples of suitable extension amplification mixes. With this technique, amplification takes place as soon as a library fragment is seeded. This technique biases less toward amplification of the shorter library fragments, because if a longer fragment is seeded first, it will begin to amplify before a shorter fragment can be seeded. The net benefit of concurrent seeding and amplification is that more larger library fragments are able to seed and amplify as compare to sequential seeding and amplification.

Any of the methods disclosed herein may also involve shifting the bias more toward the longer fragments by adding a predetermined concentration of a crowding agent to the flow cell with the adapter-tagged DNA fragments. The crowding agent may be, for example, poly(ethylene glycol) or dextran. The crowding agent affects the rate of diffusion of the fragments, and slows down both the larger and the smaller DNA fragments. When concurrent seeding and amplification is used, the presence of the crowding agent increases the chance that longer fragments can seed and amplify before shorter fragments do, and thus the bias is shifter toward amplification of the longer fragments. The presence of the crowding agent can also affect how the fragments enter into depressions 22.

The concentration of the crowding agent can be adjusted to further bias amplification toward the larger or smaller fragments, depending upon the reaction kinetics relative to the diffusion rates relative to the diffusion length bias. Crowding agent concentrations ranging from about 1% to about 40% can shift the bias. More crowding tends to shift the bias to longer fragments.

With the crowding agent, the library fragment concentration can also be adjusted to reduce polyclonality (i.e., seeding multiple library fragments in a single depression 22). Lower library fragment concentrations tend to lead to fewer depressions 22 that seed more than one library fragment. In other examples, the method further includes shifting the bias toward the shorter fragments or the longer fragments by selecting a predetermined crowding reagent. For example, poly(ethylene glycol) of a higher molecular weight has a higher viscosity and thus has a bigger impact on the mobility of the smaller fragments.

In any of the methods disclosed herein, the method may further include shifting the bias differently in different zones of the flow cell 10 by using different temperatures in the different zones during amplification of the sequential seeding and amplification or during concurrent seeding and amplification. Temperature will affect the reaction kinetics of the enzyme(s) used in amplification. For example, a colder temperature slows down the reaction kinetics, and thus shifts the bias toward the longer fragments.

The temperature can also impact fragment diffusion. In particular colder temperature slow down diffusion (whether the crowding agent is present or not), which can reduce the bias toward the shorter fragments and increase the bias toward the longer fragments.

In yet further examples, introducing the adapter-tagged DNA fragments to the flow cell 10 involves introducing the adapter-tagged DNA fragments at a slow flow rate (e.g., 100 μL/min) to shift the bias more toward the smaller fragments at a first portion of the flow cell 10 and shift the bias more toward the larger fragments at a second portion of the flow cell 10. The slow flow rate allows the faster smaller fragments to seed first, leaving the larger fragments to seed downstream.

Other parameters that may be used to bias the amplification include: buffer selection, annealing efficiency, enzyme concentration, PCR enhancers and additives, helix de-stabilizers or additives, PCR cycling conditions, DNA template quality, concentration of PCR components, PCR enzymes, proofreading polymers, time, cycling parameters, primer length, magnesium ion concentration, and/or depletion of PCR reagents. While several of these parameters relate to PCR, it is believed that they may impact clustering in a similar manner.

Still another example method for biasing the amplification toward longer fragments is shown in FIG. 5A through FIG. 5E. The flow cell 10 with the patterned structure 17D is used in this example method.

The method generally includes introducing a plurality of library fragments 36, 38, 40, 42 into a flow cell 10 including depressions 22 separated by interstitial regions 24; and primers 32A, 32B immobilized in the depressions 22, whereby at least some of the plurality of library fragments 36, 38, 40, 42 are seeded, by the primers 32A, 32B, in at least some of the depressions 22 (FIG. 5A); removing unseeded library fragments from the flow cell 10; introducing a crowding agent solution 44 into the flow cell 10, the crowding solution including: a crowding agent and a salt, whereby the seeded at least some of the plurality of library fragments 36, 38, 40, 42 are compacted within the at least some of the depressions 22 (FIG. 5B); and simultaneously i) heating the flow cell 10 to a dehybridization temperature and ii) flowing additional solution 44 through the flow cell 10, thereby removing a portion of the seeded at least some of the plurality of library fragments 40, 42 having a fragment length below a predetermined value (FIG. 5C).

The library templates 36, 38, 40, 42 used in the method shown in the FIG. 5 series may be prepared using any suitable method, including the example described herein.

In this example method, sequential seeding and amplification is used, and thus the plurality of library fragments (including fragments 36, 38, 40, 42) may be introduced into the flow cell 10 for seeding, without the enzymes for amplification. As shown in FIG. 5A, both shorter fragments 40, 42 and longer fragments 36, 38 will seed.

Once a suitable time frame for seeding has taken place, a wash solution may be transported through the flow cell 10 to remove any unseeded library fragments (not shown). An example of the wash solution is an aqueous solution including a buffer agent (e.g., Tris), a salt (e.g., sodium chloride, sodium citrate, etc.), a surfactant (e.g., TWEEN polysorbates), and/or a chelating agent (e.g., EDTA). In one example, the wash solution includes water, the salt at a concentration ranging from about 25 mM to about 50 mM, the surfactant in an amount ranging from about 0.01 wt % to about 0.1 wt %, and optionally the chelating agent.

Once seeding has taken place and unseeded library fragments have been removed, a crowding agent solution 44 is introduced into the flow cell 10 (FIG. 5B). The crowding agent solution 44 includes at least a crowding agent and a salt. In an example, the crowding agent is selected from the group consisting of poly(ethylene glycol) and dextran; and the salt is selected from the group consisting of sodium chloride (0.5 M or 1 M). In some instances, the crowding agent solution 44 also includes a liquid carrier, such as water.

Within the flow cell 10, the crowding agent solution 44 compresses the seeded library fragments 36, 38, 40, 42. While all of the fragments 36, 38, 40, 42 are compressed, the larger fragments 36, 38 tend to form aggregates. The compression of the fragments 36, 38, 40, 42 is shown in FIG. 5B.

The method then involves simultaneously heating the flow cell 10 to a dehybridization temperature and flowing additional crowding agent solution 44 through the flow cell 10, thereby removing a portion of the seeded at least some of the plurality of library fragments 40, 42 having a fragment length below a predetermined value (FIG. 5C). In an example, the dehybridization temperature ranges from about 70° C. to about 90° C. At this temperature, the library fragments 36, 38, 40, 42 dehybridize from the primers 32A, 32B. The larger library fragments 36, 38 remain aggregated, and the smaller library fragments 40, 42 equilibrate out of the aggregate, and thus can be removed with the additional crowding agent solution 44 that is flowed through the flow cell 10. This is shown in FIG. 5C.

The library fragments 40, 42 that are removed have a fragment length that is below a predetermined value. In one example, this value is below 150 bp. In another example, this value is below 200 bp. The crowding agent solution 44 can be tuned, via the concentration of the crowding agent, to remove fragments of a desired size. As examples, the concentration of the crowding agent may range from about 1% to about 40%, where a lower percentage increases the size of the fragments, e.g., 40, 42, that are to be removed, and a higher percentage decreases the size of the fragments that are to be removed.

The method then includes lowering a temperature of the flow cell 10 to below the dehybridization temperature (FIG. 5D); and introducing a buffer solution 46 into the flow cell 10, whereby a second portion of the seeded at least some of the plurality of library fragments 36, 38 having a fragment length above the predetermined value remain seeded.

As the additional crowding agent solution 44 continues to flow through the flow cell 10, the temperature of the flow cell 10 is then reduced to below the dehybridization temperature. The reduction in temperature in the presence of the crowding agent solution 44 allows the larger fragments 36, 38 to rehybridize, thus keeping the larger library fragments 36, 38 in the depressions 22. This is shown in FIG. 5D. In one example, the lower temperature ranges from about 25° C. to about 65° C.

A buffer solution 46, such as a triethylamine (TEA) buffer, that does not contain the crowding agent 44 is then introduced into the flow cell 32, as shown in FIG. 5D. The remaining seeded library fragments 36, 38 are no longer compressed and thus expand within the depressions 22, as shown in FIG. 5E.

The method then includes initiating amplification of the second portion of the seeded at least some of the plurality of library fragments 36, 38. In this example, amplification may be initiated by introducing the amplification mix.

In any of the examples set forth herein, amplification results in the formation of clusters of forward and reverse strands in the depressions 22 or along the lane 12. The forward or reverse strands may be cleaved using a cleaving agent that is targeted to a cleavage site (e.g., uracil, 8-oxoguanine) of one type of strand. The remaining strand are then sequenced. In one example, sequencing by synthesis (SBS) is performed by introducing sequencing primers followed by an incorporation mix including labeled nucleotides. Optical imaging may be used to detect each instance of nucleotide incorporation along the amplified library fragments.

To further illustrate the present disclosure, examples are given herein. It is to be understood that these examples are provided for illustrative purposes and are not to be construed as limiting the scope of the present disclosure.

### EXAMPLES

### Example 1

To demonstrate the amplification on a standard patterned flow cell, six DNA samples were prepared (e.g., tagged with adapters) and were introduced onto a commercially available patterned flow cell substrate that did not include the density gradient.

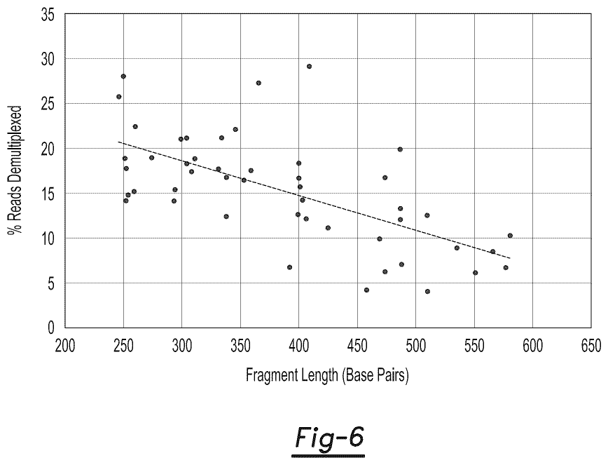

The DNA samples were subjected to bridge amplification and subsequently sequenced using sequencing by synthesis. A graphical representation of the metrics of the sequencing operation is shown in FIG. 6, with fragment length (in base pairs) being shown on the X axis and the percentage of reads demultiplexed being shown on the Y axis. As can be seen, a higher percentage of reads was demultiplexed for the shorter fragment lengths (e.g., having around 250 base pairs), relative to the longer fragment lengths (e.g., having around 575 base pairs).

These results indicate that the standard patterned flow cell, without the variations in architecture or method conditions set forth herein, preferentially bias amplification of the shorter fragments. While the results are not shown herein, similar data with different primer densities shifted these curves toward larger fragments.

### Example 2

Three different DNA fragment libraries were prepared having an average insert size of 450 base pairs, 550 base pairs, and 850 base pairs. Each of these libraries was respectively introduced into a flow cell having depression diameters of 280 nm, 320 nm, and 360 nm.

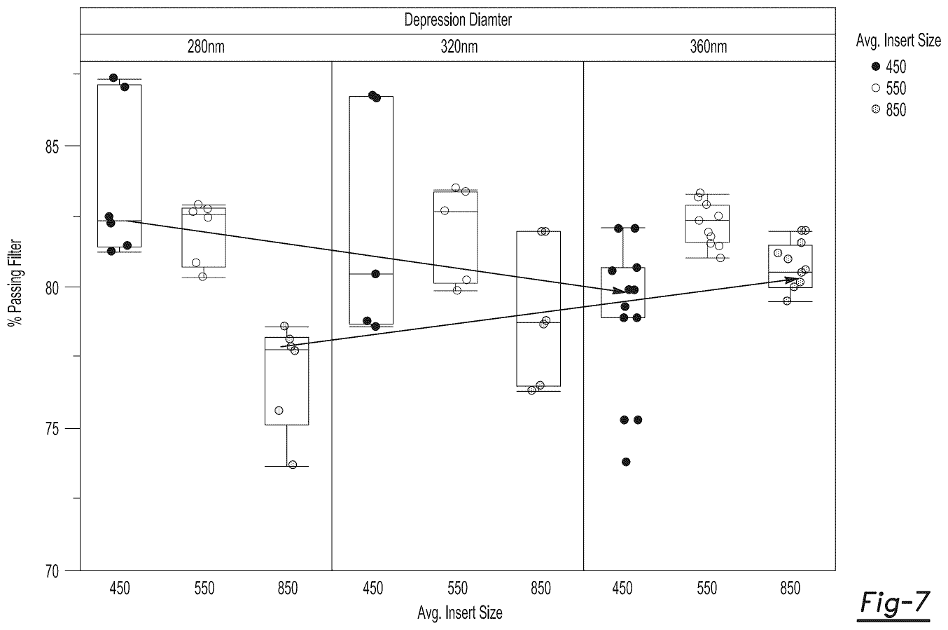

In the respective flow cells, each of the DNA fragment libraries were subjected to bridge amplification and subsequently sequenced using sequencing by synthesis. The sequencing data collected included passing filter (% PF) (percentage), and the results are shown in FIG. 7. Passing filter (PF) is the metric used to describe clusters which pass a chastity threshold and are used for further processing and analysis of sequencing data. The % PF calculation involves the application of a chastity filter to each cluster.

These results indicate that the accessibility of the primer lawn in the larger diameter depressions compared to the primer lawn in the smaller depressions shift the amplification bias to the larger fragments.

### Example 3

In this example, 10 wt % of poly(ethylene glycol) was added to three different clustering experiments to determine its effect on fragment bias. Two examples involved the sequential seeding and amplification workflow. A third example involved the concurrent seeding and amplification workflow. The presence of the PEG shifted the base pair bias by 10 base pairs in each of the examples.

### ADDITIONAL NOTES

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

Reference throughout the specification to “one example”, “another example”, “an example”, and so forth, means that a particular element (e.g., feature, structure, and/or characteristic) described in connection with the example is included in at least one example described herein, and may or may not be present in other examples. In addition, it is to be understood that the described elements for any example may be combined in any suitable manner in the various examples unless the context clearly dictates otherwise.

What is claimed is:

1. A flow cell, comprising:
a substrate;
a polymeric hydrogel positioned over at least a portion of the substrate; and
a plurality of oligonucleotide primers attached to the polymeric hydrogel, wherein the plurality of oligonucleotide primers defines a density gradient across the substrate.

2. The flow cell as defined in claim 1, wherein:
the substrate includes a plurality of depressions defined therein that are separated by interstitial regions;
the polymeric hydrogel at least partially fills each of the plurality of depressions; and
a diameter of at least some depressions in the plurality of depressions is varied to define distinct regions of the density gradient.

3. The flow cell as defined in claim 2, wherein at least two distinct regions of the density gradient are defined, and wherein each of the two distinct regions has a different primer density.

4. The flow cell as defined in claim 1, wherein a thickness of the polymeric hydrogel is varied across at least a portion of the substrate to define distinct regions of the density gradient.

5. The flow cell as defined in claim 1, wherein at least two distinct regions of the density gradient are defined, and wherein each of the two distinct regions has a different functional group density.

6. The flow cell as defined in claim 1, wherein a porosity of the polymeric hydrogel is varied across at least a portion of the substrate to define distinct regions of the density gradient.

7. A method of preparing a flow cell, comprising:
depositing a polymeric hydrogel over at least portion of a substrate; and
attaching a plurality of oligonucleotide primers to at least a portion of the polymeric hydrogel, such that the plurality of oligonucleotide primers forms a density gradient across the substrate.

8. The method as defined in claim 7, wherein:
the depositing of the polymeric hydrogel is controlled to generate a polymeric hydrogel thickness that is varied across at least a portion of the substrate; and
attaching the plurality of oligonucleotide primers to the at least the portion of the polymeric hydrogel defines distinct regions of the density gradient within the at least the portion of the substrate.

9. The method as defined in claim 8, wherein at least two distinct regions of the density gradient are defined, and wherein each of the distinct regions has a different primer density.

10. The method as defined in claim 7, wherein:
the polymeric hydrogel is a first polymeric hydrogel having a first porosity;
the at least the portion of the substrate is a first portion; and
the method further comprises depositing a second polymeric hydrogel having a second porosity that is different from the first porosity over a second portion of the substrate.

11. The method as defined in claim 7, wherein prior to depositing the polymeric hydrogel, the method further comprises patterning the substrate to include a plurality of depressions that are separated by interstitial regions, wherein a diameter of at least some depressions in the plurality of depressions is varied to define distinct regions of the density gradient.

12. The method as defined in claim 11, wherein at least two distinct regions of the density gradient are defined, and wherein each of the distinct regions has a different primer density.

13. The method as defined in claim 7, wherein during the attaching of the plurality of oligonucleotide primers to the at least the portion of the polymeric hydrogel, the method further comprises controlling a temperature of the substrate to facilitate formation of the density gradient.

14. The method as defined in claim 7, wherein during the attaching of the plurality of oligonucleotide primers to the at least the portion of the polymeric hydrogel, the method further comprises controlling a flow rate of a primer mixture including the plurality of oligonucleotide primers to facilitate formation of the density gradient.

15. The method as defined in claim 7, wherein during the attaching of the plurality of oligonucleotide primers to the at least the portion of the polymeric hydrogel, the method further comprises controlling a concentration of a primer mixture including the plurality of oligonucleotide primers to facilitate formation of the density gradient.

16. A method for biasing DNA insert size, comprising:
selecting a target fragment length to be amplified using adapter-tagged DNA fragments;
introducing the adapter-tagged DNA fragments to a flow cell including primers; and
based on the selection of the target fragment length, one of:
initiating sequential seeding and amplification of the adapter-tagged DNA fragments to shift a bias toward shorter fragments of the adapter-tagged DNA fragments; or
initiating concurrent seeding and amplification of the adapter-tagged DNA fragments to shift the bias toward longer fragments of the adapter-tagged DNA fragments.

17. The method as defined in claim 16, further comprising shifting the bias more toward the shorter fragments or the longer fragments by adding a predetermined concentration of a crowding agent to the flow cell with the adapter-tagged DNA fragments.

18. The method as defined in claim 16, further comprising shifting the bias more toward the larger fragments by lowering a temperature during sequential seeding and amplification or during concurrent seeding and amplification.

19. The method as defined in claim 16, further comprising shifting the bias differently in different zones of the flow cell by using different temperatures in the different zones during amplification of the sequential seeding and amplification or during concurrent seeding and amplification.

20. The method as defined in claim 16, wherein introducing the adapter-tagged DNA fragments to the flow cell involves introducing the adapter-tagged DNA fragments at a slow flow rate to shift the bias more toward the smaller fragments at a first portion of the flow cell and to shift the bias more toward the larger fragments at a second portion of the flow cell.

21. A method for biasing DNA insert size, comprising:
introducing a plurality of library fragments into a flow cell including:
depressions separated by interstitial regions; and
primers immobilized in the depressions, whereby at least some of the plurality of library fragments are seeded, by the primers, in at least some of the depressions;
removing unseeded library fragments from the flow cell;
introducing a crowding agent solution into the flow cell, the crowding agent solution including:
a crowding agent; and
a salt, whereby the seeded at least some of the plurality of library fragments are compacted within the at least some of the depressions; and
simultaneously heating the flow cell to a dehybridization temperature and flowing additional crowding agent solution through the flow cell, thereby removing a portion of the seeded at least some of the plurality of library fragments having a fragment length below a predetermined value.

22. The method as defined in claim 21, further comprising:
lowering a temperature of the flow cell to below the dehybridization temperature; and
introducing a buffer solution into the flow cell, whereby a second portion of the seeded at least some of the plurality of library fragments having a fragment length above the predetermined value remain seeded.

23. The method as defined in claim 22, further comprising initiating amplification of the second portion of the seeded at least some of the plurality of library fragments.

24. The method as defined in claim 22, wherein:
the crowding agent is selected from the group consisting of poly(ethylene glycol) and dextran; and
the salt is sodium chloride.

25. The method as defined in claim 22, wherein:
the dehybridization temperature ranges from about 70° C. to about 90° C.; and
the lower temperature ranges from about 25° C. to about 65° C.
